SEQUENCE LISTING (232 sequences)

SEQ ID NO: 1 - DNA, 74 bp, synthetic construct
  source 1..74: mol_type = other DNA
gccttcaaacgtacatgcgccagtcgaaggaaaaaaaaaacacatctgacgctgccgacgatagggataacagg

SEQ ID NO: 2 - DNA, 74 bp, synthetic construct
  source 1..74: mol_type = other DNA
ggcaaacgaaacattggcacacagcacagcaaaaaaaaaacacatctgacgctgccgacgatagggataacagg

SEQ ID NO: 3 - DNA, 46 bp, synthetic construct
  source 1..46: mol_type = other DNA
ccttggcacccgagaattccaagttcgtcggcagcgtcagatgtgt

SEQ ID NO: 4 - DNA, 47 bp, synthetic construct
  source 1..47: mol_type = other DNA
tcgtgcgatattttcctacgcaaagnnactttaggcggtcctagcaa

SEQ ID NO: 5 - DNA, 47 bp, synthetic construct
  source 1..47: mol_type = other DNA
cggttttcttaaggagatgcacacgnnactttaggcggtcctagcaa

SEQ ID NO: 6 - DNA, 47 bp, synthetic construct
  source 1..47: mol_type = other DNA
acagttgacaaatgttccgagctccnnactttaggcggtcctagcaa

SEQ ID NO: 7 - DNA, 47 bp, synthetic construct
  source 1..47: mol_type = other DNA
caggagtacgatttgtatagcgcctnnactttaggcggtcctagcaa

SEQ ID NO: 8 - DNA, 47 bp, synthetic construct
  source 1..47: mol_type = other DNA
cgcatttacggaacattgtggacacnnactttaggcggtcctagcaa

SEQ ID NO: 9 - DNA, 47 bp, synthetic construct
  source 1..47: mol_type = other DNA
ttcgaacttatatgcactgtccagcnnactttaggcggtcctagcaa

SEQ ID NO: 10 - DNA, 47 bp, synthetic construct
  source 1..47: mol_type = other DNA
cggtctgtgtgcaacgactaacttannactttaggcggtcctagcaa

SEQ ID NO: 11 - DNA, 47 bp, synthetic construct
  source 1..47: mol_type = other DNA
gaggctcaggtatggatctacagcannactttaggcggtcctagcaa

SEQ ID NO: 12 - DNA, 47 bp, synthetic construct
  source 1..47: mol_type = other DNA
tggtctatcttagaactggtggctcnnactttaggcggtcctagcaa

SEQ ID NO: 13 - DNA, 47 bp, synthetic construct
  source 1..47: mol_type = other DNA
gacttatggccttcagaaggattcgnnactttaggcggtcctagcaa

SEQ ID NO: 14 - DNA, 47 bp, synthetic construct
  source 1..47: mol_type = other DNA
ggtaactgcgcatagttggctctatnnactttaggcggtcctagcaa

SEQ ID NO: 15 - DNA, 47 bp, synthetic construct
  source 1..47: mol_type = other DNA
ttagacgagcaggtttcttgcctatnnactttaggcggtcctagcaa

SEQ ID NO: 16 - DNA, 47 bp, synthetic construct
  source 1..47: mol_type = other DNA
gcacactctgaaaactagcctcacannactttaggcggtcctagcaa

SEQ ID NO: 17 - DNA, 47 bp, synthetic construct
  source 1..47: mol_type = other DNA
tggcatactacctacgtgttgactgnnactttaggcggtcctagcaa

SEQ ID NO: 18 - DNA, 47 bp, synthetic construct
  source 1..47: mol_type = other DNA
gatgagaagccgcggtatacttaccnnactttaggcggtcctagcaa

SEQ ID NO: 19 - DNA, 47 bp, synthetic construct
  source 1..47: mol_type = other DNA
cgttcgtttatgtcgaagtacacgtnnactttaggcggtcctagcaa

SEQ ID NO: 20 - DNA, 47 bp, synthetic construct
  source 1..47: mol_type = other DNA
ctacaacgcagattacaacctcagtnnactttaggcggtcctagcaa

SEQ ID NO: 21 - DNA, 47 bp, synthetic construct
  source 1..47: mol_type = other DNA
ctataacggtgaggccaaactttggnnactttaggcggtcctagcaa

SEQ ID NO: 22 - DNA, 47 bp, synthetic construct
  source 1..47: mol_type = other DNA
cgccttctatcattaacacacctccnnactttaggcggtcctagcaa

SEQ ID NO: 23 - DNA, 47 bp, synthetic construct
  source 1..47: mol_type = other DNA
agcaggtgataggattgactctcctnnactttaggcggtcctagcaa

SEQ ID NO: 24 - DNA, 47 bp, synthetic construct
  source 1..47: mol_type = other DNA
tacaccatcaattcgcattctgtggnnactttaggcggtcctagcaa

SEQ ID NO: 25 - DNA, 47 bp, synthetic construct
  source 1..47: mol_type = other DNA
tattgaatacttccgctcaagagccnnactttaggcggtcctagcaa

SEQ ID NO: 26 - DNA, 47 bp, synthetic construct
  source 1..47: mol_type = other DNA
tgcatcgtacactcttgtttgaacgnnactttaggcggtcctagcaa

SEQ ID NO: 27 - DNA, 47 bp, synthetic construct
  source 1..47: mol_type = other DNA
ccttaccggatggattttatgagccnnactttaggcggtcctagcaa

SEQ ID NO: 28 - DNA, 47 bp, synthetic construct
  source 1..47: mol_type = other DNA
atgtgtcaactacgacgagtgctagnnactttaggcggtcctagcaa

SEQ ID NO: 29 - DNA, 47 bp, synthetic construct
  source 1..47: mol_type = other DNA
gccaccttaacacgcgatgatattgnnactttaggcggtcctagcaa

SEQ ID NO: 30 - DNA, 47 bp, synthetic construct
  source 1..47: mol_type = other DNA
gagtcgaatatccaccaccgtatggnnactttaggcggtcctagcaa

SEQ ID NO: 31 - DNA, 47 bp, synthetic construct
  source 1..47: mol_type = other DNA
gttcatggtacaggcttctttacggnnactttaggcggtcctagcaa

SEQ ID NO: 32 - DNA, 47 bp, synthetic construct
  source 1..47: mol_type = other DNA
ggcagtttggccagtcttgaaatcannactttaggcggtcctagcaa

SEQ ID NO: 33 - DNA, 47 bp, synthetic construct
  source 1..47: mol_type = other DNA
atcggctttagtctttcacgctaatnnactttaggcggtcctagcaa

SEQ ID NO: 34 - DNA, 47 bp, synthetic construct
  source 1..47: mol_type = other DNA
gagttcataacgattccgcctgacannactttaggcggtcctagcaa

SEQ ID NO: 35 - DNA, 47 bp, synthetic construct
  source 1..47: mol_type = other DNA
gctctcttacaatgttgatggcctannactttaggcggtcctagcaa

SEQ ID NO: 36 - DNA, 47 bp, synthetic construct
  source 1..47: mol_type = other DNA
gcctatgcgagcttccaataaaaggnnactttaggcggtcctagcaa

SEQ ID NO: 37 - DNA, 47 bp, synthetic construct
  source 1..47: mol_type = other DNA
ggctgagtcacacatcgttaccattnnactttaggcggtcctagcaa

SEQ ID NO: 38 - DNA, 47 bp, synthetic construct
  source 1..47: mol_type = other DNA
tcgccggttaagaagacactagcttnnactttaggcggtcctagcaa

SEQ ID NO: 39 - DNA, 47 bp, synthetic construct
  source 1..47: mol_type = other DNA
gtatatgttcgcgcacggtgtagacnnactttaggcggtcctagcaa

SEQ ID NO: 40 - DNA, 47 bp, synthetic construct
  source 1..47: mol_type = other DNA
cggtggtaggtgatacttctgtaccnnactttaggcggtcctagcaa

SEQ ID NO: 41 - DNA, 47 bp, synthetic construct
  source 1..47: mol_type = other DNA
tggacggtttgaaagttcctcgactnnactttaggcggtcctagcaa

SEQ ID NO: 42 - DNA, 47 bp, synthetic construct
  source 1..47: mol_type = other DNA
agaacgacgtactttcatgcaggacnnactttaggcggtcctagcaa

SEQ ID NO: 43 - DNA, 47 bp, synthetic construct
  source 1..47: mol_type = other DNA
cggaccagtggtatacaggagaagcnnactttaggcggtcctagcaa

SEQ ID NO: 44 - DNA, 47 bp, synthetic construct
  source 1..47: mol_type = other DNA
cttgaggtagcaattcttcggacaannactttaggcggtcctagcaa

SEQ ID NO: 45 - DNA, 47 bp, synthetic construct
  source 1..47: mol_type = other DNA
ttattccgtggaaatccgcattaccnnactttaggcggtcctagcaa

SEQ ID NO: 46 - DNA, 47 bp, synthetic construct
  source 1..47: mol_type = other DNA
gtgtttacgtgcttcgtttggatccnnactttaggcggtcctagcaa

SEQ ID NO: 47 - DNA, 47 bp, synthetic construct
  source 1..47: mol_type = other DNA
actgcattggcgaccgtttaactaannactttaggcggtcctagcaa

SEQ ID NO: 48 - DNA, 47 bp, synthetic construct
  source 1..47: mol_type = other DNA
tatcggttcaatccggctaaaggctnnactttaggcggtcctagcaa

SEQ ID NO: 49 - DNA, 47 bp, synthetic construct
  source 1..47: mol_type = other DNA
ggaaacacgttttcagataaccacgnnactttaggcggtcctagcaa

SEQ ID NO: 50 - DNA, 47 bp, synthetic construct
  source 1..47: mol_type = other DNA
atacgttacgttagtggctggttccnnactttaggcggtcctagcaa

SEQ ID NO: 51 - DNA, 47 bp, synthetic construct
  source 1..47: mol_type = other DNA
caaaccagtaagatgaaccgctagtnnactttaggcggtcctagcaa

SEQ ID NO: 52 - DNA, 47 bp, synthetic construct
  source 1..47: mol_type = other DNA
ggcgtatattgagttagacacgtccnnactttaggcggtcctagcaa

SEQ ID NO: 53 - DNA, 47 bp, synthetic construct
  source 1..47: mol_type = other DNA
tactaccgtagcttgcctcctttacnnactttaggcggtcctagcaa

SEQ ID NO: 54 - DNA, 47 bp, synthetic construct
  source 1..47: mol_type = other DNA
gttagctggcaatcaggatccatttnnactttaggcggtcctagcaa

SEQ ID NO: 55 - DNA, 47 bp, synthetic construct
  source 1..47: mol_type = other DNA
accggtagtaactgaatttagtgcgnnactttaggcggtcctagcaa

SEQ ID NO: 56 - DNA, 47 bp, synthetic construct
  source 1..47: mol_type = other DNA
ggtgcttggaaatagctagacgatgnnactttaggcggtcctagcaa

SEQ ID NO: 57 - DNA, 47 bp, synthetic construct
  source 1..47: mol_type = other DNA
tggttacctaatctagcaccacgtannactttaggcggtcctagcaa

SEQ ID NO: 58 - DNA, 47 bp, synthetic construct
  source 1..47: mol_type = other DNA
tttgtggcaaccatccgttcagatannactttaggcggtcctagcaa

SEQ ID NO: 59 - DNA, 47 bp, synthetic construct
  source 1..47: mol_type = other DNA
gagctttcaaactcattgcaactgcnnactttaggcggtcctagcaa

SEQ ID NO: 60 - DNA, 47 bp, synthetic construct
  source 1..47: mol_type = other DNA
tgtatgcaaaactctcctgtgcatgnnactttaggcggtcctagcaa

SEQ ID NO: 61 - DNA, 47 bp, synthetic construct
  source 1..47: mol_type = other DNA
tttgaccaagatggttcgtatcaccnnactttaggcggtcctagcaa

SEQ ID NO: 62 - DNA, 47 bp, synthetic construct
  source 1..47: mol_type = other DNA
gtagcgtatctattagcggagccgannactttaggcggtcctagcaa

SEQ ID NO: 63 - DNA, 47 bp, synthetic construct
  source 1..47: mol_type = other DNA
cgttggtcactgtaaggacgaatggnnactttaggcggtcctagcaa

SEQ ID NO: 64 - DNA, 47 bp, synthetic construct
  source 1..47: mol_type = other DNA
cgccaatccttagtaaaagacgcatnnactttaggcggtcctagcaa

SEQ ID NO: 65 - DNA, 47 bp, synthetic construct
  source 1..47: mol_type = other DNA
tggttaggttagtctccgtaaacctnnactttaggcggtcctagcaa

SEQ ID NO: 66 - DNA, 47 bp, synthetic construct
  source 1..47: mol_type = other DNA
tttcggaatagacgcttctaatgcannactttaggcggtcctagcaa

SEQ ID NO: 67 - DNA, 47 bp, synthetic construct
  source 1..47: mol_type = other DNA
gtcttggaacggattctctcggatannactttaggcggtcctagcaa

SEQ ID NO: 68 - DNA, 90 bp, synthetic construct
  source 1..90: mol_type = other DNA
ggcagcgtcagatgtgttcgtgcgatattttcctacgcaaagaaaaagcttagatgattttgaacgtgtgcgcctgttatccctatcgtc

SEQ ID NO: 69 - DNA, 90 bp, synthetic construct
  source 1..90: mol_type = other DNA
ggcagcgtcagatgtgtcggttttcttaaggagatgcacacgaaaaaactcgtcttggtaattcgtacgtcccctgttatccctatcgtc

SEQ ID NO: 70 - DNA, 90 bp, synthetic construct
  source 1..90: mol_type = other DNA
ggcagcgtcagatgtgtacagttgacaaatgttccgagctccaaaaaccttggccttatcacatactggaagcctgttatccctatcgtc

SEQ ID NO: 71 - DNA, 90 bp, synthetic construct
  source 1..90: mol_type = other DNA
ggcagcgtcagatgtgtcaggagtacgatttgtatagcgcctaaaaagtcctcgaatgaagcaatgcctaatcctgttatccctatcgtc

SEQ ID NO: 72 - DNA, 90 bp, synthetic construct
  source 1..90: mol_type = other DNA
ggcagcgtcagatgtgtcgcatttacggaacattgtggacacaaaaaagcaggagctaccatgagtttctttcctgttatccctatcgtc

SEQ ID NO: 73 - DNA, 90 bp, synthetic construct
  source 1..90: mol_type = other DNA
ggcagcgtcagatgtgtttcgaacttatatgcactgtccagcaaaaaaattactggagtgccttatgccgtgcctgttatccctatcgtc

SEQ ID NO: 74 - DNA, 90 bp, synthetic construct
  source 1..90: mol_type = other DNA
ggcagcgtcagatgtgtcggtctgtgtgcaacgactaacttaaaaaagctcgtaataattgctccaaaccgacctgttatccctatcgtc

SEQ ID NO: 75 - DNA, 90 bp, synthetic construct
  source 1..90: mol_type = other DNA
ggcagcgtcagatgtgtgaggctcaggtatggatctacagcaaaaaagcaattcatagcgttaatcggtcgtcctgttatccctatcgtc

SEQ ID NO: 76 - DNA, 90 bp, synthetic construct
  source 1..90: mol_type = other DNA
ggcagcgtcagatgtgttggtctatcttagaactggtggctcaaaaagccttagattcttaagcaccggattcctgttatccctatcgtc

SEQ ID NO: 77 - DNA, 90 bp, synthetic construct
  source 1..90: mol_type = other DNA
ggcagcgtcagatgtgtgacttatggccttcagaaggattcgaaaaatttcgatgaattaggtcaagcctcgcctgttatccctatcgtc

SEQ ID NO: 78 - DNA, 90 bp, synthetic construct
  source 1..90: mol_type = other DNA
ggcagcgtcagatgtgtggtaactgcgcatagttggctctataaaaaccttaggatagccgaatcttgacgacctgttatccctatcgtc

SEQ ID NO: 79 - DNA, 90 bp, synthetic construct
  source 1..90: mol_type = other DNA
ggcagcgtcagatgtgtttagacgagcaggtttcttgcctataaaaagccgagtagaagttgcgattttggtcctgttatccctatcgtc

SEQ ID NO: 80 - DNA, 90 bp, synthetic construct
  source 1..90: mol_type = other DNA
ggcagcgtcagatgtgtgcacactctgaaaactagcctcacaaaaaaaagtccgttttaatgagacgactggcctgttatccctatcgtc

SEQ ID NO: 81 - DNA, 90 bp, synthetic construct
  source 1..90: mol_type = other DNA
ggcagcgtcagatgtgttggcatactacctacgtgttgactgaaaaagcatcacatgcgtaggtctcaaaggcctgttatccctatcgtc

SEQ ID NO: 82 - DNA, 90 bp, synthetic construct
  source 1..90: mol_type = other DNA
ggcagcgtcagatgtgtgatgagaagccgcggtatacttaccaaaaaaagagcatctgctaaatgacgatcccctgttatccctatcgtc

SEQ ID NO: 83 - DNA, 90 bp, synthetic construct
  source 1..90: mol_type = other DNA
ggcagcgtcagatgtgtcgttcgtttatgtcgaagtacacgtaaaaagtaaatggacttgcacgtaacctgtcctgttatccctatcgtc

SEQ ID NO: 84 - DNA, 90 bp, synthetic construct
  source 1..90: mol_type = other DNA
ggcagcgtcagatgtgtctacaacgcagattacaacctcagtaaaaagcttagatgattttgaacgtgtgcgcctgttatccctatcgtc

SEQ ID NO: 85 - DNA, 90 bp, synthetic construct
  source 1..90: mol_type = other DNA
ggcagcgtcagatgtgtctataacggtgaggccaaactttggaaaaaactcgtcttggtaattcgtacgtcccctgttatccctatcgtc

SEQ ID NO: 86 - DNA, 90 bp, synthetic construct
  source 1..90: mol_type = other DNA
ggcagcgtcagatgtgtcgccttctatcattaacacacctccaaaaaccttggccttatcacatactggaagcctgttatccctatcgtc

SEQ ID NO: 87 - DNA, 90 bp, synthetic construct
  source 1..90: mol_type = other DNA
ggcagcgtcagatgtgtagcaggtgataggattgactctcctaaaaagtcctcgaatgaagcaatgcctaatcctgttatccctatcgtc

SEQ ID NO: 88 - DNA, 90 bp, synthetic construct
  source 1..90: mol_type = other DNA
ggcagcgtcagatgtgttacaccatcaattcgcattctgtggaaaaaagcaggagctaccatgagtttctttcctgttatccctatcgtc

SEQ ID NO: 89 - DNA, 90 bp, synthetic construct
  source 1..90: mol_type = other DNA
ggcagcgtcagatgtgttattgaatacttccgctcaagagccaaaaaaattactggagtgccttatgccgtgcctgttatccctatcgtc

SEQ ID NO: 90 - DNA, 90 bp, synthetic construct
  source 1..90: mol_type = other DNA
ggcagcgtcagatgtgttgcatcgtacactcttgtttgaacgaaaaagctcgtaataattgctccaaaccgacctgttatccctatcgtc

SEQ ID NO: 91 - DNA, 90 bp, synthetic construct
  source 1..90: mol_type = other DNA
ggcagcgtcagatgtgtccttaccggatggattttatgagccaaaaagcaattcatagcgttaatcggtcgtcctgttatccctatcgtc

SEQ ID NO: 92 - DNA, 90 bp, synthetic construct
  source 1..90: mol_type = other DNA
ggcagcgtcagatgtgtatgtgtcaactacgacgagtgctagaaaaagccttagattcttaagcaccggattcctgttatccctatcgtc

SEQ ID NO: 93 - DNA, 90 bp, synthetic construct
  source 1..90: mol_type = other DNA
ggcagcgtcagatgtgtgccaccttaacacgcgatgatattgaaaaatttcgatgaattaggtcaagcctcgcctgttatccctatcgtc

SEQ ID NO: 94 - DNA, 90 bp, synthetic construct
  source 1..90: mol_type = other DNA
ggcagcgtcagatgtgtgagtcgaatatccaccaccgtatggaaaaaccttaggatagccgaatcttgacgacctgttatccctatcgtc

SEQ ID NO: 95 - DNA, 90 bp, synthetic construct
  source 1..90: mol_type = other DNA
ggcagcgtcagatgtgtgttcatggtacaggcttctttacggaaaaagccgagtagaagttgcgattttggtcctgttatccctatcgtc

SEQ ID NO: 96 - DNA, 90 bp, synthetic construct
  source 1..90: mol_type = other DNA
ggcagcgtcagatgtgtggcagtttggccagtcttgaaatcaaaaaaaagtccgttttaatgagacgactggcctgttatccctatcgtc

SEQ ID NO: 97 - DNA, 90 bp, synthetic construct
  source 1..90: mol_type = other DNA
ggcagcgtcagatgtgtatcggctttagtctttcacgctaataaaaagcatcacatgcgtaggtctcaaaggcctgttatccctatcgtc

SEQ ID NO: 98 - DNA, 90 bp, synthetic construct
  source 1..90: mol_type = other DNA
ggcagcgtcagatgtgtgagttcataacgattccgcctgacaaaaaaaagagcatctgctaaatgacgatcccctgttatccctatcgtc

SEQ ID NO: 99 - DNA, 90 bp, synthetic construct
  source 1..90: mol_type = other DNA
ggcagcgtcagatgtgtgctctcttacaatgttgatggcctaaaaaagtaaatggacttgcacgtaacctgtcctgttatccctatcgtc

SEQ ID NO: 100 - DNA, 90 bp, synthetic construct
  source 1..90: mol_type = other DNA
ggcagcgtcagatgtgtgcctatgcgagcttccaataaaaggaaaaagcttagatgattttgaacgtgtgcgcctgttatccctatcgtc

SEQ ID NO: 101 - DNA, 90 bp, synthetic construct
  source 1..90: mol_type = other DNA
ggcagcgtcagatgtgtggctgagtcacacatcgttaccattaaaaaactcgtcttggtaattcgtacgtcccctgttatccctatcgtc

SEQ ID NO: 102 - DNA, 90 bp, synthetic construct
  source 1..90: mol_type = other DNA
ggcagcgtcagatgtgttcgccggttaagaagacactagcttaaaaaccttggccttatcacatactggaagcctgttatccctatcgtc

SEQ ID NO: 103 - DNA, 90 bp, synthetic construct
  source 1..90: mol_type = other DNA
ggcagcgtcagatgtgtgtatatgttcgcgcacggtgtagacaaaaagtcctcgaatgaagcaatgcctaatcctgttatccctatcgtc

SEQ ID NO: 104 - DNA, 90 bp, synthetic construct
  source 1..90: mol_type = other DNA
ggcagcgtcagatgtgtcggtggtaggtgatacttctgtaccaaaaaagcaggagctaccatgagtttctttcctgttatccctatcgtc

SEQ ID NO: 105 - DNA, 90 bp, synthetic construct
  source 1..90: mol_type = other DNA
ggcagcgtcagatgtgttggacggtttgaaagttcctcgactaaaaaaattactggagtgccttatgccgtgcctgttatccctatcgtc

SEQ ID NO: 106 - DNA, 90 bp, synthetic construct
  source 1..90: mol_type = other DNA
ggcagcgtcagatgtgtagaacgacgtactttcatgcaggacaaaaagctcgtaataattgctccaaaccgacctgttatccctatcgtc

SEQ ID NO: 107 - DNA, 90 bp, synthetic construct
  source 1..90: mol_type = other DNA
ggcagcgtcagatgtgtcggaccagtggtatacaggagaagcaaaaagcaattcatagcgttaatcggtcgtcctgttatccctatcgtc

SEQ ID NO: 108 - DNA, 90 bp, synthetic construct
  source 1..90: mol_type = other DNA
ggcagcgtcagatgtgtcttgaggtagcaattcttcggacaaaaaaagccttagattcttaagcaccggattcctgttatccctatcgtc

SEQ ID NO: 109 - DNA, 90 bp, synthetic construct
  source 1..90: mol_type = other DNA
ggcagcgtcagatgtgtttattccgtggaaatccgcattaccaaaaatttcgatgaattaggtcaagcctcgcctgttatccctatcgtc

SEQ ID NO: 110 - DNA, 90 bp, synthetic construct
  source 1..90: mol_type = other DNA
ggcagcgtcagatgtgtgtgtttacgtgcttcgtttggatccaaaaaccttaggatagccgaatcttgacgacctgttatccctatcgtc

SEQ ID NO: 111 - DNA, 90 bp, synthetic construct
  source 1..90: mol_type = other DNA
ggcagcgtcagatgtgtactgcattggcgaccgtttaactaaaaaaagccgagtagaagttgcgattttggtcctgttatccctatcgtc

SEQ ID NO: 112 - DNA, 90 bp, synthetic construct
  source 1..90: mol_type = other DNA
ggcagcgtcagatgtgttatcggttcaatccggctaaaggctaaaaaaagtccgttttaatgagacgactggcctgttatccctatcgtc

SEQ ID NO: 113 - DNA, 90 bp, synthetic construct
  source 1..90: mol_type = other DNA
ggcagcgtcagatgtgtggaaacacgttttcagataaccacgaaaaagcatcacatgcgtaggtctcaaaggcctgttatccctatcgtc

SEQ ID NO: 114 - DNA, 90 bp, synthetic construct
  source 1..90: mol_type = other DNA
ggcagcgtcagatgtgtatacgttacgttagtggctggttccaaaaaaagagcatctgctaaatgacgatcccctgttatccctatcgtc

SEQ ID NO: 115 - DNA, 90 bp, synthetic construct
  source 1..90: mol_type = other DNA
ggcagcgtcagatgtgtcaaaccagtaagatgaaccgctagtaaaaagtaaatggacttgcacgtaacctgtcctgttatccctatcgtc

SEQ ID NO: 116 - DNA, 90 bp, synthetic construct
  source 1..90: mol_type = other DNA
ggcagcgtcagatgtgtggcgtatattgagttagacacgtccaaaaagcttagatgattttgaacgtgtgcgcctgttatccctatcgtc

SEQ ID NO: 117 - DNA, 90 bp, synthetic construct
  source 1..90: mol_type = other DNA
ggcagcgtcagatgtgttactaccgtagcttgcctcctttacaaaaaactcgtcttggtaattcgtacgtcccctgttatccctatcgtc

SEQ ID NO: 118 - DNA, 90 bp, synthetic construct
  source 1..90: mol_type = other DNA
ggcagcgtcagatgtgtgttagctggcaatcaggatccatttaaaaaccttggccttatcacatactggaagcctgttatccctatcgtc

SEQ ID NO: 119 - DNA, 90 bp, synthetic construct
  source 1..90: mol_type = other DNA
ggcagcgtcagatgtgtaccggtagtaactgaatttagtgcgaaaaagtcctcgaatgaagcaatgcctaatcctgttatccctatcgtc

SEQ ID NO: 120 - DNA, 90 bp, synthetic construct
  source 1..90: mol_type = other DNA
ggcagcgtcagatgtgtggtgcttggaaatagctagacgatgaaaaaagcaggagctaccatgagtttctttcctgttatccctatcgtc

SEQ ID NO: 121 - DNA, 90 bp, synthetic construct
  source 1..90: mol_type = other DNA
ggcagcgtcagatgtgttggttacctaatctagcaccacgtaaaaaaaattactggagtgccttatgccgtgcctgttatccctatcgtc

SEQ ID NO: 122 - DNA, 90 bp, synthetic construct
  source 1..90: mol_type = other DNA
ggcagcgtcagatgtgttttgtggcaaccatccgttcagataaaaaagctcgtaataattgctccaaaccgacctgttatccctatcgtc

SEQ ID NO: 123 - DNA, 90 bp, synthetic construct
  source 1..90: mol_type = other DNA
ggcagcgtcagatgtgtgagctttcaaactcattgcaactgcaaaaagcaattcatagcgttaatcggtcgtcctgttatccctatcgtc

SEQ ID NO: 124 - DNA, 90 bp, synthetic construct
  source 1..90: mol_type = other DNA
ggcagcgtcagatgtgttgtatgcaaaactctcctgtgcatgaaaaagccttagattcttaagcaccggattcctgttatccctatcgtc

SEQ ID NO: 125 - DNA, 90 bp, synthetic construct
  source 1..90: mol_type = other DNA
ggcagcgtcagatgtgttttgaccaagatggttcgtatcaccaaaaatttcgatgaattaggtcaagcctcgcctgttatccctatcgtc

SEQ ID NO: 126 - DNA, 90 bp, synthetic construct
  source 1..90: mol_type = other DNA
ggcagcgtcagatgtgtgtagcgtatctattagcggagccgaaaaaaccttaggatagccgaatcttgacgacctgttatccctatcgtc

SEQ ID NO: 127 - DNA, 90 bp, synthetic construct
  source 1..90: mol_type = other DNA
ggcagcgtcagatgtgtcgttggtcactgtaaggacgaatggaaaaagccgagtagaagttgcgattttggtcctgttatccctatcgtc

SEQ ID NO: 128 - DNA, 90 bp, synthetic construct
  source 1..90: mol_type = other DNA
ggcagcgtcagatgtgtcgccaatccttagtaaaagacgcataaaaaaagtccgttttaatgagacgactggcctgttatccctatcgtc

SEQ ID NO: 129 - DNA, 90 bp, synthetic construct
  source 1..90: mol_type = other DNA
ggcagcgtcagatgtgttggttaggttagtctccgtaaacctaaaaagcatcacatgcgtaggtctcaaaggcctgttatccctatcgtc

SEQ ID NO: 130 - DNA, 90 bp, synthetic construct
  source 1..90: mol_type = other DNA
ggcagcgtcagatgtgttttcggaatagacgcttctaatgcaaaaaaaagagcatctgctaaatgacgatcccctgttatccctatcgtc

SEQ ID NO: 131 - DNA, 90 bp, synthetic construct
  source 1..90: mol_type = other DNA
ggcagcgtcagatgtgtgtcttggaacggattctctcggataaaaaagtaaatggacttgcacgtaacctgtcctgttatccctatcgtc

SEQ ID NO: 132 - DNA, 20 bp, synthetic construct
  source 1..20: mol_type = other DNA
actccactactactcactct

SEQ ID NO: 133 - DNA, 20 bp, synthetic construct
  source 1..20: mol_type = other DNA
atcctcactacatcatccac

SEQ ID NO: 134 - DNA, 20 bp, synthetic construct
  source 1..20: mol_type = other DNA
tacatccaactccctacact

SEQ ID NO: 135 - DNA, 20 bp, synthetic construct
  source 1..20: mol_type = other DNA
tcttcttcatccacaccttc

SEQ ID NO: 136 - DNA, 20 bp, synthetic construct
  source 1..20: mol_type = other DNA
tcctaacaaccaactactcc

SEQ ID NO: 137 - DNA, 65 bp, synthetic construct
  source 1..65: mol_type = other DNA
ggagtagttggttgttaggagcttagatgattttgaacgtgtgcgggagtagttggttgttagga

SEQ ID NO: 138 - DNA, 65 bp, synthetic construct
  source 1..65: mol_type = other DNA
ggagtagttggttgttaggaactcgtcttggtaattcgtacgtccggagtagttggttgttagga

SEQ ID NO: 139 - DNA, 65 bp, synthetic construct
  source 1..65: mol_type = other DNA
gtggatgatgtagtgaggatccttggccttatcacatactggaaggtggatgatgtagtgaggat

SEQ ID NO: 140 - DNA, 65 bp, synthetic construct
  source 1..65: mol_type = other DNA
gtggatgatgtagtgaggatgtcctcgaatgaagcaatgcctaatgtggatgatgtagtgaggat

SEQ ID NO: 141 - DNA, 65 bp, synthetic construct
  source 1..65: mol_type = other DNA
agagtgagtagtagtggagtagcaggagctaccatgagtttctttagagtgagtagtagtggagt

SEQ ID NO: 142 - DNA, 65 bp, synthetic construct
  source 1..65: mol_type = other DNA
agagtgagtagtagtggagtaattactggagtgccttatgccgtgagagtgagtagtagtggagt

SEQ ID NO: 143 - DNA, 65 bp, synthetic construct
  source 1..65: mol_type = other DNA
gtggatgatgtagtgaggatgctcgtaataattgctccaaaccgagtggatgatgtagtgaggat

SEQ ID NO: 144 - DNA, 65 bp, synthetic construct
  source 1..65: mol_type = other DNA
agtgtagggagttggatgtagcaattcatagcgttaatcggtcgtagtgtagggagttggatgta

SEQ ID NO: 145 - DNA, 65 bp, synthetic construct
  source 1..65: mol_type = other DNA
ggagtagttggttgttaggagccttagattcttaagcaccggattggagtagttggttgttagga

SEQ ID NO: 146 - DNA, 65 bp, synthetic construct
  source 1..65: mol_type = other DNA
gtggatgatgtagtgaggattttcgatgaattaggtcaagcctcggtggatgatgtagtgaggat

SEQ ID NO: 147 - DNA, 65 bp, synthetic construct
  source 1..65: mol_type = other DNA
agtgtagggagttggatgtaccttaggatagccgaatcttgacgaagtgtagggagttggatgta

SEQ ID NO: 148 - DNA, 65 bp, synthetic construct
  source 1..65: mol_type = other DNA
agagtgagtagtagtggagtgccgagtagaagttgcgattttggtagagtgagtagtagtggagt

SEQ ID NO: 149 - DNA, 65 bp, synthetic construct
  source 1..65: mol_type = other DNA
agtgtagggagttggatgtaaagtccgttttaatgagacgactggagtgtagggagttggatgta

SEQ ID NO: 150 - DNA, 65 bp, synthetic construct
  source 1..65: mol_type = other DNA
agtgtagggagttggatgtagcatcacatgcgtaggtctcaaaggagtgtagggagttggatgta

SEQ ID NO: 151 - DNA, 65 bp, synthetic construct
  source 1..65: mol_type = other DNA
agagtgagtagtagtggagtaagagcatctgctaaatgacgatccagagtgagtagtagtggagt

SEQ ID NO: 152 - DNA, 65 bp, synthetic construct
  source 1..65: mol_type = other DNA
ggagtagttggttgttaggagtaaatggacttgcacgtaacctgtggagtagttggttgttagga

SEQ ID NO: 153 - DNA, 65 bp, synthetic construct
  source 1..65: mol_type = other DNA
agagtgagtagtagtggagtgcttagatgattttgaacgtgtgcgagagtgagtagtagtggagt

SEQ ID NO: 154 - DNA, 65 bp, synthetic construct
  source 1..65: mol_type = other DNA
agtgtagggagttggatgtaactcgtcttggtaattcgtacgtccagtgtagggagttggatgta

SEQ ID NO: 155 - DNA, 65 bp, synthetic construct
  source 1..65: mol_type = other DNA
agagtgagtagtagtggagtccttggccttatcacatactggaagagagtgagtagtagtggagt

SEQ ID NO: 156 - DNA, 65 bp, synthetic construct
  source 1..65: mol_type = other DNA
agtgtagggagttggatgtagtcctcgaatgaagcaatgcctaatagtgtagggagttggatgta

SEQ ID NO: 157 - DNA, 65 bp, synthetic construct
  source 1..65: mol_type = other DNA
agtgtagggagttggatgtaagcaggagctaccatgagtttctttagtgtagggagttggatgta

SEQ ID NO: 158 - DNA, 65 bp, synthetic construct
  source 1..65: mol_type = other DNA
gtggatgatgtagtgaggataattactggagtgccttatgccgtggtggatgatgtagtgaggat

SEQ ID NO: 159 - DNA, 65 bp, synthetic construct; identical to SEQ ID NO: 143
  source 1..65: mol_type = other DNA

SEQ ID NO: 160 - DNA, 65 bp, synthetic construct; identical to SEQ ID NO: 144
  source 1..65: mol_type = other DNA

SEQ ID NO: 161 - DNA, 65 bp, synthetic construct; identical to SEQ ID NO: 145
  source 1..65: mol_type = other DNA

SEQ ID NO: 162 - DNA, 65 bp, synthetic construct
  source 1..65: mol_type = other DNA
ggagtagttggttgttaggatttcgatgaattaggtcaagcctcgggagtagttggttgttagga

SEQ ID NO: 163 - DNA, 65 bp, synthetic construct
  source 1..65: mol_type = other DNA
agagtgagtagtagtggagtccttaggatagccgaatcttgacgaagagtgagtagtagtggagt

SEQ ID NO: 164 - DNA, 65 bp, synthetic construct
  source 1..65: mol_type = other DNA
ggagtagttggttgttaggagccgagtagaagttgcgattttggtggagtagttggttgttagga

SEQ ID NO: 165 - DNA, 65 bp, synthetic construct
  source 1..65: mol_type = other DNA
gtggatgatgtagtgaggataagtccgttttaatgagacgactgggtggatgatgtagtgaggat

SEQ ID NO: 166 - DNA, 65 bp, synthetic construct
  source 1..65: mol_type = other DNA
ggagtagttggttgttaggagcatcacatgcgtaggtctcaaaggggagtagttggttgttagga

SEQ ID NO: 167 - DNA, 65 bp, synthetic construct; identical to SEQ ID NO: 151
  source 1..65: mol_type = other DNA

SEQ ID NO: 168 - DNA, 65 bp, synthetic construct
  source 1..65: mol_type = other DNA
gtggatgatgtagtgaggatgtaaatggacttgcacgtaacctgtgtggatgatgtagtgaggat

SEQ ID NO: 169 - DNA, 65 bp, synthetic construct
  source 1..65: mol_type = other DNA
agagtgagtagtagtggagttcgtgcgatattttcctacgcaaagagagtgagtagtagtggagt

SEQ ID NO: 170 - DNA, 65 bp, synthetic construct
  source 1..65: mol_type = other DNA
agagtgagtagtagtggagtcggttttcttaaggagatgcacacgagagtgagtagtagtggagt

SEQ ID NO: 171 - DNA, 65 bp, synthetic construct
  source 1..65: mol_type = other DNA
agagtgagtagtagtggagtacagttgacaaatgttccgagctccagagtgagtagtagtggagt

SEQ ID NO: 172 - DNA, 65 bp, synthetic construct
  source 1..65: mol_type = other DNA
agagtgagtagtagtggagtcaggagtacgatttgtatagcgcctagagtgagtagtagtggagt

SEQ ID NO: 173 - DNA, 65 bp, synthetic construct
  source 1..65: mol_type = other DNA
agagtgagtagtagtggagtcgcatttacggaacattgtggacacagagtgagtagtagtggagt

SEQ ID NO: 174 - DNA, 65 bp, synthetic construct
  source 1..65: mol_type = other DNA
agagtgagtagtagtggagtttcgaacttatatgcactgtccagcagagtgagtagtagtggagt

SEQ ID NO: 175 - DNA, 65 bp, synthetic construct
  source 1..65: mol_type = other DNA
agagtgagtagtagtggagtcggtctgtgtgcaacgactaacttaagagtgagtagtagtggagt

SEQ ID NO: 176 - DNA, 65 bp, synthetic construct
  source 1..65: mol_type = other DNA
agagtgagtagtagtggagtgaggctcaggtatggatctacagcaagagtgagtagtagtggagt

SEQ ID NO: 177 - DNA, 65 bp, synthetic construct
  source 1..65: mol_type = other DNA
agagtgagtagtagtggagttggtctatcttagaactggtggctcagagtgagtagtagtggagt

SEQ ID NO: 178 - DNA, 65 bp, synthetic construct
  source 1..65: mol_type = other DNA
agagtgagtagtagtggagtgacttatggccttcagaaggattcgagagtgagtagtagtggagt

SEQ ID NO: 179 - DNA, 65 bp, synthetic construct
  source 1..65: mol_type = other DNA
agagtgagtagtagtggagtggtaactgcgcatagttggctctatagagtgagtagtagtggagt

SEQ ID NO: 180 - DNA, 65 bp, synthetic construct
  source 1..65: mol_type = other DNA
agagtgagtagtagtggagtttagacgagcaggtttcttgcctatagagtgagtagtagtggagt

SEQ ID NO: 181 - DNA, 65 bp, synthetic construct
  source 1..65: mol_type = other DNA
agagtgagtagtagtggagtgcacactctgaaaactagcctcacaagagtgagtagtagtggagt

SEQ ID NO: 182 - DNA, 65 bp, synthetic construct
  source 1..65: mol_type = other DNA
agagtgagtagtagtggagttggcatactacctacgtgttgactgagagtgagtagtagtggagt

SEQ ID NO: 183 - DNA, 65 bp, synthetic construct
  source 1..65: mol_type = other DNA
agagtgagtagtagtggagtgatgagaagccgcggtatacttaccagagtgagtagtagtggagt

SEQ ID NO: 184 - DNA, 65 bp, synthetic construct
  source 1..65: mol_type = other DNA
agagtgagtagtagtggagtcgttcgtttatgtcgaagtacacgtagagtgagtagtagtggagt

SEQ ID NO: 185 - DNA, 65 bp, synthetic construct
  source 1..65: mol_type = other DNA
gtggatgatgtagtgaggatctacaacgcagattacaacctcagtgtggatgatgtagtgaggat

SEQ ID NO: 186 - DNA, 65 bp, synthetic construct
  source 1..65: mol_type = other DNA
gtggatgatgtagtgaggatctataacggtgaggccaaactttgggtggatgatgtagtgaggat

SEQ ID NO: 187 - DNA, 65 bp, synthetic construct
  source 1..65: mol_type = other DNA
gtggatgatgtagtgaggatcgccttctatcattaacacacctccgtggatgatgtagtgaggat

SEQ ID NO: 188 - DNA, 65 bp, synthetic construct
  source 1..65: mol_type = other DNA
gtggatgatgtagtgaggatagcaggtgataggattgactctcctgtggatgatgtagtgaggat

SEQ ID NO: 189 - DNA, 65 bp, synthetic construct
  source 1..65: mol_type = other DNA
gtggatgatgtagtgaggattacaccatcaattcgcattctgtgggtggatgatgtagtgaggat

SEQ ID NO: 190 - DNA, 65 bp, synthetic construct
  source 1..65: mol_type = other DNA
gtggatgatgtagtgaggattattgaatacttccgctcaagagccgtggatgatgtagtgaggat

SEQ ID NO: 191 - DNA, 65 bp, synthetic construct
  source 1..65: mol_type = other DNA
gtggatgatgtagtgaggattgcatcgtacactcttgtttgaacggtggatgatgtagtgaggat

SEQ ID NO: 192 - DNA, 65 bp, synthetic construct
  source 1..65: mol_type = other DNA
gtggatgatgtagtgaggatccttaccggatggattttatgagccgtggatgatgtagtgaggat

SEQ ID NO: 193 - DNA, 65 bp, synthetic construct
  source 1..65: mol_type = other DNA
gtggatgatgtagtgaggatatgtgtcaactacgacgagtgctaggtggatgatgtagtgaggat

SEQ ID NO: 194 - DNA, 65 bp, synthetic construct
  source 1..65: mol_type = other DNA
gtggatgatgtagtgaggatgccaccttaacacgcgatgatattggtggatgatgtagtgaggat

SEQ ID NO: 195 - DNA, 65 bp, synthetic construct
  source 1..65: mol_type = other DNA
gtggatgatgtagtgaggatgagtcgaatatccaccaccgtatgggtggatgatgtagtgaggat

SEQ ID NO: 196 - DNA, 65 bp, synthetic construct
  source 1..65: mol_type = other DNA
gtggatgatgtagtgaggatgttcatggtacaggcttctttacgggtggatgatgtagtgaggat

SEQ ID NO: 197 - DNA, 65 bp, synthetic construct
  source 1..65: mol_type = other DNA
gtggatgatgtagtgaggatggcagtttggccagtcttgaaatcagtggatgatgtagtgaggat

SEQ ID NO: 198 - DNA, 65 bp, synthetic construct
  source 1..65: mol_type = other DNA
gtggatgatgtagtgaggatatcggctttagtctttcacgctaatgtggatgatgtagtgaggat

SEQ ID NO: 199 - DNA, 65 bp, synthetic construct
  source 1..65: mol_type = other DNA
gtggatgatgtagtgaggatgagttcataacgattccgcctgacagtggatgatgtagtgaggat

SEQ ID NO: 200 - DNA, 65 bp, synthetic construct
  source 1..65: mol_type = other DNA
gtggatgatgtagtgaggatgctctcttacaatgttgatggcctagtggatgatgtagtgaggat

SEQ ID NO: 201 - DNA, 65 bp, synthetic construct
  source 1..65: mol_type = other DNA
agtgtagggagttggatgtagcctatgcgagcttccaataaaaggagtgtagggagttggatgta

SEQ ID NO: 202 - DNA, 65 bp, synthetic construct
  source 1..65: mol_type = other DNA
agtgtagggagttggatgtaggctgagtcacacatcgttaccattagtgtagggagttggatgta

SEQ ID NO: 203 - DNA, 65 bp, synthetic construct
  source 1..65: mol_type = other DNA
agtgtagggagttggatgtatcgccggttaagaagacactagcttagtgtagggagttggatgta

SEQ ID NO: 204 - DNA, 65 bp, synthetic construct
  source 1..65: mol_type = other DNA
agtgtagggagttggatgtagtatatgttcgcgcacggtgtagacagtgtagggagttggatgta

SEQ ID NO: 205 - DNA, 65 bp, synthetic construct
  source 1..65: mol_type = other DNA
agtgtagggagttggatgtacggtggtaggtgatacttctgtaccagtgtagggagttggatgta

SEQ ID NO: 206 - DNA, 65 bp, synthetic construct
  source 1..65: mol_type = other DNA
agtgtagggagttggatgtatggacggtttgaaagttcctcgactagtgtagggagttggatgta

SEQ ID NO: 207 - DNA, 65 bp, synthetic construct
  source 1..65: mol_type = other DNA
agtgtagggagttggatgtaagaacgacgtactttcatgcaggacagtgtagggagttggatgta

SEQ ID NO: 208 - DNA, 65 bp, synthetic construct
  source 1..65: mol_type = other DNA
agtgtagggagttggatgtacggaccagtggtatacaggagaagcagtgtagggagttggatgta

SEQ ID NO: 209 - DNA, 65 bp, synthetic construct
  source 1..65: mol_type = other DNA
agtgtagggagttggatgtacttgaggtagcaattcttcggacaaagtgtagggagttggatgta

SEQ ID NO: 210 - DNA, 65 bp, synthetic construct
  source 1..65: mol_type = other DNA
agtgtagggagttggatgtattattccgtggaaatccgcattaccagtgtagggagttggatgta

SEQ ID NO: 211 - DNA, 65 bp, synthetic construct
  source 1..65: mol_type = other DNA
agtgtagggagttggatgtagtgtttacgtgcttcgtttggatccagtgtagggagttggatgta

SEQ ID NO: 212 - DNA, 65 bp, synthetic construct
  source 1..65: mol_type = other DNA
agtgtagggagttggatgtaactgcattggcgaccgtttaactaaagtgtagggagttggatgta

SEQ ID NO: 213 - DNA, 65 bp, synthetic construct
  source 1..65: mol_type = other DNA
agtgtagggagttggatgtatatcggttcaatccggctaaaggctagtgtagggagttggatgta

SEQ ID NO: 214 - DNA, 65 bp, synthetic construct
  source 1..65: mol_type = other DNA
agtgtagggagttggatgtaggaaacacgttttcagataaccacgagtgtagggagttggatgta

SEQ ID NO: 215 - DNA, 65 bp, synthetic construct
  source 1..65: mol_type = other DNA
agtgtagggagttggatgtaatacgttacgttagtggctggttccagtgtagggagttggatgta

SEQ ID NO: 216 - DNA, 65 bp, synthetic construct
  source 1..65: mol_type = other DNA
agtgtagggagttggatgtacaaaccagtaagatgaaccgctagtagtgtagggagttggatgta

SEQ ID NO: 217 - DNA, 65 bp, synthetic construct
  source 1..65: mol_type = other DNA
ggagtagttggttgttaggaggcgtatattgagttagacacgtccggagtagttggttgttagga

SEQ ID NO: 218 - DNA, 65 bp, synthetic construct
  source 1..65: mol_type = other DNA
ggagtagttggttgttaggatactaccgtagcttgcctcctttacggagtagttggttgttagga

SEQ ID NO: 219 - DNA, 65 bp, synthetic construct
  source 1..65: mol_type = other DNA
ggagtagttggttgttaggagttagctggcaatcaggatccatttggagtagttggttgttagga

SEQ ID NO: 220 - DNA, 65 bp, synthetic construct
  source 1..65: mol_type = other DNA
ggagtagttggttgttaggaaccggtagtaactgaatttagtgcgggagtagttggttgttagga

SEQ ID NO: 221 - DNA, 65 bp, synthetic construct
  source 1..65: mol_type = other DNA
ggagtagttggttgttaggaggtgcttggaaatagctagacgatgggagtagttggttgttagga

SEQ ID NO: 222 - DNA, 65 bp, synthetic construct
  source 1..65: mol_type = other DNA
ggagtagttggttgttaggatggttacctaatctagcaccacgtaggagtagttggttgttagga

SEQ ID NO: 223 - DNA, 65 bp, synthetic construct
  source 1..65: mol_type = other DNA
ggagtagttggttgttaggatttgtggcaaccatccgttcagataggagtagttggttgttagga

SEQ ID NO: 224 - DNA, 65 bp, synthetic construct
  source 1..65: mol_type = other DNA
ggagtagttggttgttaggagagctttcaaactcattgcaactgcggagtagttggttgttagga

SEQ ID NO: 225 - DNA, 65 bp, synthetic construct
  source 1..65: mol_type = other DNA
ggagtagttggttgttaggatgtatgcaaaactctcctgtgcatgggagtagttggttgttagga

SEQ ID NO: 226 - DNA, 65 bp, synthetic construct
  source 1..65: mol_type = other DNA
ggagtagttggttgttaggatttgaccaagatggttcgtatcaccggagtagttggttgttagga

SEQ ID NO: 227 - DNA, 65 bp, synthetic construct
  source 1..65: mol_type = other DNA
ggagtagttggttgttaggagtagcgtatctattagcggagccgaggagtagttggttgttagga

SEQ ID NO: 228 - DNA, 65 bp, synthetic construct
  source 1..65: mol_type = other DNA
ggagtagttggttgttaggacgttggtcactgtaaggacgaatggggagtagttggttgttagga

SEQ ID NO: 229 - DNA, 65 bp, synthetic construct
  source 1..65: mol_type = other DNA
ggagtagttggttgttaggacgccaatccttagtaaaagacgcatggagtagttggttgttagga

SEQ ID NO: 230 - DNA, 65 bp, synthetic construct
  source 1..65: mol_type = other DNA
ggagtagttggttgttaggatggttaggttagtctccgtaaacctggagtagttggttgttagga

SEQ ID NO: 231 - DNA, 65 bp, synthetic construct
  source 1..65: mol_type = other DNA
ggagtagttggttgttaggatttcggaatagacgcttctaatgcaggagtagttggttgttagga

SEQ ID NO: 232 - DNA, 65 bp, synthetic construct
  source 1..65: mol_type = other DNA
ggagtagttggttgttaggagtcttggaacggattctctcggataggagtagttggttgttagga

(19) United States
(12) Patent Application Publication
(10) Pub. No.: US 2025/0011847 A1
(43) Pub. Date: Jan. 9, 2025

(54) METHODS FOR INDEXING SINGLE CELLS AND NUCLEI USING NANOBALL COMBINATORIAL IDENTIFIERS

(71) Applicant: Hu Cang, San Diego, CA (US)

(72) Inventor: Hu Cang, San Diego, CA (US)

(21) Appl. No.: 18/750,271

(22) Filed: Jun. 21, 2024

Related U.S. Application Data
(60) Provisional application No. 63/524,908, filed on Jul. 4, 2023.

(51) Int. Cl. C12Q 1/6841; C12Q 1/6874

(52) U.S. Cl. CPC C12Q 1/6841; C12Q 1/6874

(57) ABSTRACT

The present disclosure relates to a method for spatial single-cell sequencing. The method includes collecting a sample comprising a plurality of cells or nuclei. The method also includes amplifying oligos and generating a plurality of nanoballs within each of the cell or nucleus. The method also includes creating a nanoball combinatorial identifier (NCI) or a unique nanoball combinatorial identifier (UNCI) for each of the cell or nucleus based on the combination of the nanoballs. The method also includes identifying the nanoballs using both optical microscopy and next-generation sequencing (NGS)-based single-cell or single-nucleus sequencing assays. The method also includes dissociating the cells or nuclei from tissues, and the dissociated cells or nuclei are subjected to the single-cell or single-nucleus sequencing. The method also includes subsequently sequencing the nanoballs to correlate the spatial location/coordinates of each cell or nuclei with the single-cell or single-nucleus sequencing data.

### CROSS-REFERENCE TO RELATED APPLICATIONS

This application claims the benefit of U.S. Provisional Application No. 63/524,908 titled “NANOBALL COMBINATORIAL IDENTIFIERS: METHODS FOR INDEXING SINGLE-CELL AND NUCLEI” filed by the applicant on 5 Jul. 2023, which is incorporated herein by reference in its entirety.

### SEQUENCE LISTING

The application herein incorporates by reference in its entirety the sequence listing material in the XML file named Nanoball Combinatorial Identifiers.xml, created 7 Jun. 2024, and having the size of 210 kilobytes, filed with this application.

### FIELD OF THE INVENTION

Embodiments of the present invention relate to the field of spatial molecular biology and more specifically to a method for indexing single-cell and nuclei using nanoball combinatorial identifiers.

### DESCRIPTION OF THE RELATED ART

In the field of single-cell sequencing and spatial biology, there are many next-generation sequencing (NGS)-based single-cell technologies that can perform various types of single-cell sequencing including, single-cell RNA sequencing (sc-RNA-seq) providing detailed information on gene expression at the single-cell level, single-cell assay for transposase-accessible chromatin using sequencing (sc-ATAC-seq) giving insights into chromatin accessibility, cellular indexing of transcriptomes and epitopes by sequencing (sc-CITE-seq) allowing for the simultaneous measurement of ribonucleic acid (RNA) and protein markers, single-cell methylation sequencing (sc-Methylation-seq) analyzing deoxyribonucleic acid (DNA) methylation patterns, single-cell Chromatin immunoprecipitation followed by sequencing (sc-ChIP-seq) identifying protein-DNA interactions.

While such technologies provide rich data on various aspects of cellular states, they lack the ability to map these states within their spatial context in tissues. This spatial information is crucial for understanding cells interaction within their native environments. There are many challenges associated with NGS-based methods. Traditional NGS-based methods often fail to preserve or provide spatial information, which means they cannot tell where exactly in the tissue a particular cell type or state is located. Some methods use fabricated microstructures to capture or label nucleotides in cells. However, the cost associated with fabricating these microstructures can be prohibitively high.

Another popular technique is microfluidics-based single-cell spatial assays, the assays involve manipulating tiny volumes of fluids to capture, label or analyze cells. While promising in providing some spatial context, they often do not achieve the resolution needed to distinguish individual cells accurately.

Yet another popular technique includes imaging-based spatial biology platforms including, MERFISH (Multiplexed Error-Robust Fluorescence In Situ Hybridization), Seq-FISH (Sequential Fluorescence In Situ Hybridization), Fluorescent in situ SEQuencing (FISSEQ) and Hybridization-based in situ sequencing (HybISS). While imaging-based platforms are effective at profiling RNA, they are restricted in their ability to provide comprehensive genome-wide coverage. Additionally, such techniques primarily focus on RNA and struggle with sequencing other cellular modalities, such as chromatin accessibility (ATAC-seq), DNA methylation, gene mutations, clonal expansion, and epigenetics.

US patent application 20150267251 provides an imaging-based spatial transcriptomics technology for detecting and/or quantifying nucleic acids in cells, tissues, organs, or organisms.

US patent application 20230227895 discloses another imaging-based spatial transcriptomics method.

U.S. patent application Ser. No. 11/098,303 discloses another imaging-based spatial transcriptomics method.

U.S. patent application Ser. No. 11/492,662B2 discloses another imaging-based spatial multi-omics method for in situ transcriptomics and proteomics.

There is a clear limitation of the currently used techniques that they do not combine the single-cell-resolution, multi-modal capabilities, and genome-wide coverage of NGS with the spatial context provided by imaging, which is urgently needed. There is need of integrated approach that enables researchers to achieve genome-wide, multi-modal single-cell sequencing with spatial information, greatly enhancing our understanding of cellular states within tissues.

Therefore, the present invention provides a method for indexing single-cell and nuclei using nanoball combinatorial identifiers, enabling correlating microscopy images of the cells or nuclei with their genomics, epigenomics or proteomics information.

### SUMMARY OF THE INVENTION

Embodiments of the present invention relate to a method for spatial single-cell sequencing. The method also includes steps of collecting a sample having a plurality of cells or nuclei, amplifying oligos and generating a plurality of nanoballs within each of the cells or nuclei, creating a nanoball combinatorial identifier (NCI) or a unique nanoball combinatorial identifier (UNCI) for each of the cell or nucleus based on the combination of the nanoballs, identifying the nanoballs using both optical microscopy and next-generation sequencing (NGS)-based single-cell sequencing assays, dissociating the cells or nuclei from tissues for single-cell sequencing, indexing the spatial information of each of the cell or nucleus using the combinations of the nanoballs, and subsequently sequencing the nanoballs to correlate the spatial location/coordinates of each cell or nucleus with the single-cell sequencing data. In particular, the single-cell sequencing data includes sc-RNA-seq data, sc-ATAC-seq data, sc-ChIP-seq data, sc-Methylation-seq data, and sc-clonal sequencing data

In accordance with an embodiment of the present invention, each of the cells or nuclei contains multiple oligos.

In accordance with an embodiment of the present invention, the combination of the nanoballs within each of the cells or nuclei creates a nanoball combinatorial identifier (NCI) for the cells or nuclei, and the combination of unique nanoballs serves as the unique nanoball combinatorial identifier (UNCI). Particularly, during nanoball generation, one or more unique molecular identifiers (UMIs) may be used, allowing for subsequent sequencing and counting.

Further, the nanoball combinatorial identifier (NCI) or the unique nanoball combinatorial identifier (UNCI) is detectable by both optical microscopy and single-cell sequencing, enabling the correlation of spatial coordinates of the tagged cells or nuclei with single-cell or single-nuclei sequencing data, including sc-RNA-seq, sc-ATAC-seq, sc-ChIP-seq, sc-Methylation-seq, and sc-clonal seq.

In accordance with an embodiment of the present invention, the nanoballs are generated by rolling circular amplification (RCA) through a plurality of circularized linear oligo or a plurality of padlock oligo anchored to the cells or nuclei.

In accordance with an embodiment of the present invention, the circularized linear oligo or padlock oligo could be anchored with anchor oligos.

Another embodiment of the present invention relates to a method for enabling the spatial tagging of single cells or nuclei within tissues. The method includes collecting a plurality of biological samples, performing fixation and permeabilization on the collected biological samples, employing a set of DNA oligos to stain the cells or nuclei within the tissues, amplifying each DNA oligo in situ to generate a plurality of replicas located together as the nanoballs, employing optical microscopy to perform imaging and read out the nanoballs, enabling the recording of spatial coordinates for each of the cells or nuclei, segmenting the cells or nuclei using a plurality of algorithms including, but not limited to, Otsu, watershed, and neural networks, to generate masks for each of the cells or nuclei, using the masks to identify the nanoballs within each of the cells or nuclei, subsequently dissociating the cells or nuclei using standard protocols for single-cell or single-nucleus isolation, subjecting the isolated single cells or nuclei to a plurality of single-cell or single-nucleus sequencing techniques, determining the sequence of the nanoballs within each of the cells or nuclei, enabling the correlation of genomic information with spatial coordinates, and integrating the obtained spatial and genomic data to gain valuable insights into the cellular composition and organization within tissues.

In accordance with an embodiment of the present invention, the sample may include, but is not limited to, tissues and cells.

In accordance with one embodiment of the present invention, the replicas may be located together as a set of the nanoballs containing the same sequence. Alternatively, the replicas may be located together as a set of the nanoballs containing complementary sequence.

In accordance with an embodiment of the present invention, the distribution of the nanoballs among the cells or nuclei is random, and the combination of the nanoballs creates a plurality of unique nanoball identifier for each of the cell or nucleus.

In accordance with an embodiment of the present invention, the combination of nanoballs creates the nanoball combinatorial identifier (NCI), while the combination of unique nanoballs creates the unique nanoball combinatorial identifier (UNCI).

In accordance with an embodiment of the present invention, the single-cell or single-nucleus sequencing techniques include but are not limited to droplet-based approaches or combination-based approaches or fluidics-based approaches.

In accordance with an embodiment of the present invention, the sequence of the nanoballs within each of the cell or nucleus is determined using the respective single-cell sequencing.

In accordance with an embodiment of the present invention, the sequencing of the nanoballs is performed using a single-cell ATAC-seq process which includes hybridization of a primer to the nanoball after optical imaging, extending the hybridized nanoball through a polymerase to create a double-stranded DNA and sequencing double-stranded DNA by following single-cell ATAC-seq protocols, including but not limited to 10×'s sc-ATAC-seq.

In accordance with an embodiment of the present invention, the sequencing of the nanoballs is performed using a 10×'s Chromium single-cell RNA sequencing kit. Oligos containing endonuclease restriction sites are hybridized to the nanoballs after optical imaging. Subsequently, endonucleases are applied to fragment the oligo/nanoball hybrid, allowing the fragments of the nanoballs to be captured by the beads in the Chromium sc-RNA sequencing kit.

In accordance with an embodiment of the present invention, the sequencing of the nanoballs is performed using a 10×'s Chromium fixed RNA protocol. The protocol includes hybridizing a pair of DNA oligos to the nanoballs and subsequently ligating them together, allowing the ligated oligos to be sequenced following the 10×'s Chromium fixed RNA protocol.

In accordance with an embodiment of the present invention, the sequencing of the nanoballs includes incorporating a unique molecular identifier (UMI) into the sequence of the nanoballs.

In accordance with an embodiment of the present invention, the unique molecular identifier (UMI) serves as a molecular tag that enables accurate counting of the nanoballs during sequencing.

The method further comprises dissociating the cells or nuclei using a plurality of enzymatic or mechanical processes.

The method further comprises incorporating unique molecular identifier (UMI). During counting, a plurality of identical nanoballs is distinguished and counted as separate entities that allows for correlation between the microscopy count and the sequencing count of non-unique nanoballs.

In accordance with an embodiment of the present invention, the method includes a color combinatorial coding approach to image the nanoballs.

In accordance with an embodiment of the present invention, the nanoballs are generated in situ using multiple rounds of hybridization.

In accordance with an embodiment of the present invention, the multiple rounds of hybridization for generating the nanoballs further include staining a first set of oligos with nucleic acid or DNA oligo-tagged antibodies in a sample, hybridizing a second set of oligos to the first plurality, with the second set being specific to the first set, subsequently hybridizing a third set of oligos to the second plurality and reiterating the process for a predetermined number of iterations.

In accordance with an embodiment of the present invention, the nanoballs are read out using Single cell combinatorial indexing RNA sequencing (sci-RNA-seq) kit when the nanoballs include restriction sites. The process comprises hybridization of the nanoballs containing a polyA sequence to a reverse-transcription primer (RT-primer), using a hairpin oligo to ligate to the reverse-transcription primer (RT-primer), performing elongation using the reverse-transcription primer (RT-primer) as a template, and incorporating the sci-RNA-seq barcodes to enable the generation of the sci-RNA-seq library for the nanoball readout.

The foregoing objectives of the present invention are achieved by employing methods for indexing single-cell and nuclei using nanoball combinatorial identifiers.

### BRIEF DESCRIPTION OF THE DRAWINGS

So that the manner in which the above-recited features of the present invention is understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

The invention herein will be better understood from the following description with reference to the drawings, in which:

FIG. 1 illustrates a flowchart for a method for spatial single-cell or single-nucleus sequencing in accordance with an embodiment of the present invention;

FIG. 2 illustrates a flowchart for a method for enabling the spatial tagging of the single cells or nuclei within tissues in accordance with an embodiment of the present invention;

FIG. 3A illustrates an exemplary nanoball combinatorial indexing in accordance with one embodiment of the present invention;

FIG. 3B illustrates an exemplary nanoball combinatorial indexing in accordance with another embodiment of the present invention;

FIG. 3C illustrates an exemplary nanoball combinatorial indexing in accordance with yet another embodiment of the present invention;

FIG. 4A illustrates an exemplary error correction in the nanoball combinatorial indexing in accordance with an embodiment of the present invention;

FIG. 4B illustrates an exemplary error correction in the nanoball combinatorial indexing in accordance with another embodiment of the present invention;

FIG. 5 illustrates a plurality of nanoball combinatorial identifiers index single cells in accordance with an embodiment of the present invention.

FIG. 6A illustrates an in-situ generation of the nanoball combinatorial identifiers in accordance with an embodiment of the present invention;

FIG. 6B illustrates an in-situ generation of the nanoball combinatorial identifiers in accordance with another embodiment of the present invention;

FIG. 6C illustrates an in-situ generation and a microscopic readout with fluorescence in situ hybridization of the nanoball combinatorial identifiers in accordance with yet another embodiment of the present invention;

FIG. 7A illustrates an in-situ generation of the nanoball combinatorial identifiers in accordance with an embodiment of the present invention;

FIG. 7B illustrates an in-situ generation of the nanoball combinatorial identifiers in accordance with an embodiment of the present invention;

FIG. 7C illustrates an in-situ generation of the nanoball combinatorial identifiers in accordance with an embodiment of the present invention;

FIG. 7D illustrates an in-situ generation of the nanoball combinatorial identifiers in accordance with an embodiment of the present invention;

FIG. 8A illustrates using a primer to convert single-stranded nanoballs into double strand for subsequent readout of the nanoball combinatorial identifiers with sc-ATAC-seq in accordance with one or more embodiments of the present invention;

FIG. 8B illustrates the nanoball that have been converted into double strand for sc-ATAC-seq readout in accordance with one or more embodiments of the present invention;

FIG. 9A illustrates a nanoball combinatorial identifier captured by beads with sc-ATAC-seq or sc-RNA-seq in accordance with an embodiment of the present invention, whereby the single stranded nanoball is hybridized by primers, and;

FIG. 9B illustrates a plurality of the nanoball combinatorial identifiers captured by beads with sc-ATAC-seq or sc-RNA-seq in accordance with another embodiment of the present invention, whereby nucleases are induced to bind to the double stranded region on the nanoball, and;

FIG. 9C illustrates a plurality of the nanoball combinatorial identifiers captured by beads with sc-ATAC-seq or sc-RNA-seq in accordance with yet another embodiment of the present invention, whereby the nanoball is fragmented;

FIG. 9D illustrates a plurality of the nanoball combinatorial identifiers captured by beads with sc-ATAC-seq or sc-RNA-seq in accordance with yet another embodiment of the present invention, whereby the fragments are captured by the beads that are used in the sc-RNA-seq kit for subsequent single-cell sequencing;

FIG. 10 illustrates a readout of the nanoball combinatorial identifiers with sc-RNA-seq in accordance with an embodiment of the present invention;

FIG. 11 illustrates a unique molecular identifier (UMI) for counting the nanoballs in accordance with an embodiment of the present invention;

FIG. 12A illustrates an exemplary color combinatorial coding in accordance with an embodiment of the present invention;

FIG. 12B illustrates an exemplary color combinatorial coding in accordance with another embodiment of the present invention;

FIG. 13A illustrates an in-situ generation of the nanoball combinatorial identifier via hybridization in accordance with another embodiment of the present invention;

FIG. 13B illustrates a capture of the hybridization generated nanoballs by beads used in sc-RNA-seq kit for subsequent readout of the nanoball combinatorial identifiers in accordance with an embodiment of the present invention;

FIG. 14 illustrates a readout of the nanoball combinatorial identifiers via sci-RNA-seq in accordance with an embodiment of the present invention;

FIG. 15A and FIG. 15B illustrate the microscopy readout of a plurality of the nanoball combinatorial identifiers in cells in accordance with an embodiment of the present invention;

FIG. 16 illustrates the microscopy images of a plurality of the nanoball combinatorial identifiers that index single cells in accordance with an embodiment of the present invention;

FIG. 17 illustrates the DNA sequence of an exemplary nanoball in accordance with an embodiment of the present invention.

It should be noted that the accompanying Figures are intended to present illustrations of exemplary embodiments of the present disclosure. These Figures are not intended to limit the scope of the present disclosure. It should also be noted that the accompanying Figures are not necessarily drawn to scale.

### DETAILED DESCRIPTION OF THE INVENTION

In the following detailed description, numerous specific details are set forth in order to provide a thorough understanding of the embodiment of the invention as illustrative or exemplary embodiments of the invention, specific embodiments in which the invention may be practiced are described in sufficient detail to enable those skilled in the art to practice the disclosed embodiments. However, it will be obvious to a person skilled in the art that the embodiments of the invention may be practiced with or without these specific details. In other instances, well-known methods, procedures and components have not been described in detail so as not to unnecessarily obscure aspects of the embodiments of the invention.

The following detailed description is, therefore, not to be taken in a limiting sense, and the scope of the present invention is defined by the appended claims and equivalents thereof. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. References within the specification to “one embodiment,” “an embodiment,” “embodiments,” or “one or more embodiments” are intended to indicate that a particular feature, structure, or characteristic described in connection with the embodiment is included in at least one embodiment of the present invention.

Although the terms first, second, etc. may be used herein to describe various elements, these elements should not be limited by these terms. These terms are generally only used to distinguish one element from another and do not denote any order, ranking, quantity, or importance, but rather are used to distinguish one element from another. Further, the terms “a” and “an” herein do not denote a limitation of quantity, but rather denote the presence of at least one of the referenced items.

The conditional language used herein, such as, among others, “can,” “may,” “might,” “may,” “e.g.,” and the like, unless specifically stated otherwise, or otherwise understood within the context as used, is generally intended to convey that certain embodiments include, while other embodiments do not include, certain features, elements and/or steps.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

### The Following Brief Definition of Terms Shall Apply Throughout the Present Invention

The terms “determining”, “measuring”, “evaluating”, “assessing,” “assaying,” and “analyzing” can be used interchangeably herein to refer to any form of measurement and include determining if an element is present or not. (e.g., detection). These terms can include both quantitative and/or qualitative determinations. Assessing may be relative or absolute.

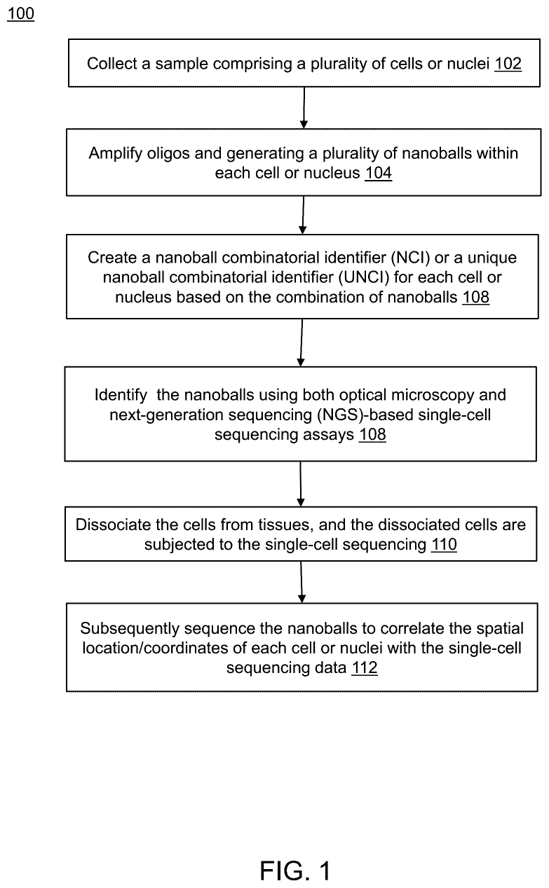

FIG. 1 illustrates a flowchart for a method 100 for spatial single-cell or single-nucleus sequencing in accordance with an embodiment of the present invention. The method 100 may comprise the following steps.

At 102, a sample comprising a plurality of cells or nuclei is collected. Each of the cells or nuclei contains multiple oligos.

At 104, oligos are amplified to generate a plurality of nanoballs within each of the cells or nuclei.

At 106, a nanoball combinatorial identifier (NCI) or a unique nanoball combinatorial identifier (UNCI) is created for each of the cells or nuclei based on the combination of the nanoballs. The combination of the nanoballs within each of the cells or nuclei creates a nanoball combinatorial identifier (NCI), and the combination of the unique nanoballs serves as the unique nanoball combinatorial identifier (UNCI).

At 108, the nanoballs are identified using both optical microscopy and next-generation sequencing (NGS)-based single-cell sequencing assays.

At 110, the cells or nuclei are dissociated from tissues. The dissociated cells or nuclei are subjected to the single-cell or single-nucleus sequencing. Further, the indexing of the spatial information of each of the cells or nuclei is performed using the combinations of the nanoballs.

At 112, the nanoballs are subsequently sequenced to correlate the spatial location/coordinates of each cells or nuclei with the single-cell sequencing data. The single-cell sequencing data includes but is not limited to single-cell-RNA-sequencing (sc-RNA-seq) data, single-cell assay for transposase-accessible chromatin using sequencing (sc-ATAC-seq) data, single-cell chromatin immunoprecipitation sequencing (sc-ChIP-seq) data, single-cell (sc)-Methylation-seq data, single-cell (sc)-clonal sequencing data.

Further, during the nanoball generation, unique molecular identifiers (UMIs) may be used to for subsequent counting non-unique nanoballs.

In accordance with an embodiment of the present invention, the nanoball combinatorial identifier (NCI) or the unique nanoball combinatorial identifier (UNCI) may be detectable by both optical microscopy and sequencing enabling the correlation of spatial coordinates of the tagged cells or nuclei with single-cell or single-nuclei sequencing data, which include, but are not limited to sc-RNA-seq, sc-ATAC-seq, sc-ChIP-seq, sc-Methylation-seq, and sc-clonal seq.

In accordance with an embodiment of the present invention, the nanoballs may be generated by rolling circular amplification (RCA) through a plurality of circularized linear oligo or a plurality of padlock oligo anchored to the cells or nuclei; the anchoring could be achieved with anchor oligos.

In accordance with an embodiment of the present invention, the NCI or UNCI, being detectable by both optical microscopy and sequencing, enables the correlation of spatial coordinates of the tagged cells or nuclei with their single-cell or single-nuclei sequencing data, which include, but are not limited to sc-RNA-seq, sc-ATAC-seq, sc-ChIP-seq, sc-Methylation-seq, and sc-clonal seq, thereby facilitating a highly efficient, low-cost, and scalable method for high-resolution mapping and analysis of complex biological system.

In a preferred embodiment, the nanoballs are generated through rolling circular amplification (RCA), wherein circularized linear oligos, also known as padlock oligos, are employed. Particularly, the padlock oligos may be anchored to cells/nuclei through anchor oligos, which hybridize with nucleic acids in the cells. Subsequently, the padlocks are circularized and amplified through RCA, resulting in the generation of the nanoballs.

In some embodiments, the method 100 may also involve the use of unique molecular identifiers (UMIs) in the nanoballs, allowing for subsequent sequencing and counting.

In some embodiments, fluorescence in situ hybridization (FISH) is employed to detect the nanoballs, and the combination of the nanoballs within each cell/nucleus defines a code space, creating a combinatorial identifier for the cell/nucleus.

In accordance with an embodiment of the present invention, the nanoballs may be read out using 10× Genomics' Chromium single-cell fixed RNA kits. This involves hybridizing the nanoballs with oligo probe pairs (left-hand side (LHS) and right-hand side (RHS)), ligating the pairs into single strands, and using unique barcodes in the LHS or RHS oligos for single-cell sequencing and counting.

In some embodiment, the nanoballs can be fragmented using a nuclease or Uracil-Specific Excision Reagent (USER) enzymes. These fragmented oligos are then captured by beads and sequenced following standard single-cell or single-nuclei sequencing assays.

In an alternative embodiment, the nanoballs may be converted into double strands by hybridizing them with another primer and extending the primer with polymerase. The double-stranded nanoballs can then be sequenced using single-cell/nucleus ATAC-seq. in some embodiments, the nanoballs may be sequenced using combinatorial indexing-based single-cell/nucleus sequencing assays.

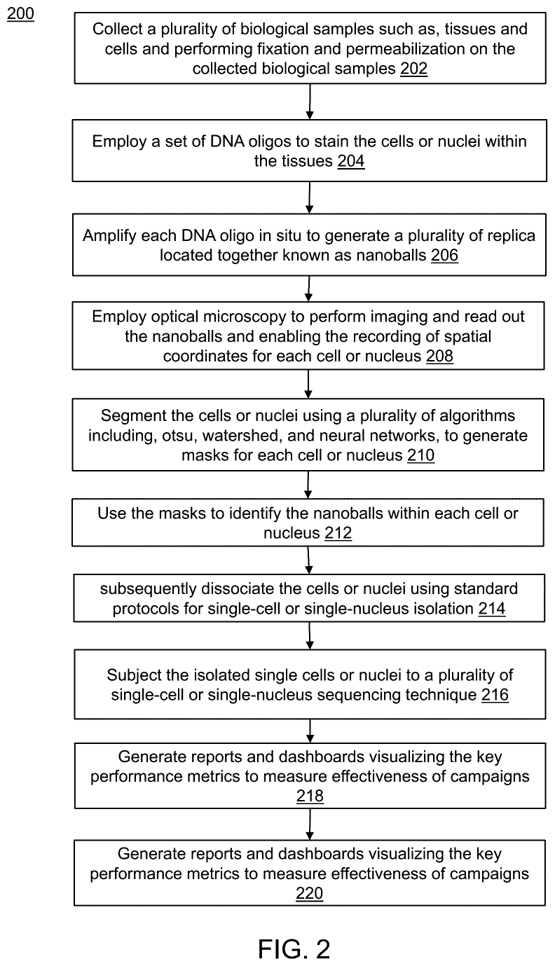

FIG. 2 illustrates a flowchart for a method 200 for enabling the spatial tagging of the single cells or nuclei within tissues in accordance with an embodiment of the present invention. The method 200 may comprise following steps.

At 202, a plurality of biological samples, such as tissues and cells, are fixed and permeabilized using standard immunohistochemistry or immunofluorescence procedures.

At 204, a set of DNA oligos is employed to stain the cells or nuclei within the tissues.

At 206, each DNA oligo is amplified in situ to generate a plurality of replicas located together as the nanoballs.

At 208, optical microscopy may be employed to image and read out the nanoballs, enabling the recording of spatial coordinates for each of the cell or nucleus.

At 210, the cells or nuclei are segmented using a plurality of algorithms including, but not limited to Otsu, watershed, and neural networks, to generate masks for each of the cells or nuclei.

At 212, the masks are used to identify the nanoballs within each of the cells or nuclei.

At 214, the cells or nuclei are subsequently dissociated using standard protocols for single-cell or single-nucleus isolation.

At 216, the isolated single cells or nuclei are subjected to a plurality of single-cell or single-nucleus sequencing techniques.

At 218, the sequence of the nanoballs within each of the cells or nuclei is determined to enable the correlation of the genomic information with spatial coordinates of the cells or nuclei.

At 220, the obtained spatial and genomic data is integrated to gain valuable insights into the cellular composition and organization within tissues.

The method 200 may further comprise dissociating the cells or nuclei using a plurality of enzymatic or mechanical process.

The method 200 may be compatible with essentially any single-nuclei or single-cell library preparation or sequencing method, enabling the incorporation of spatial coordinates into the sequencing data. Further, the method 200 may provide valuable spatial information for the sequenced cells or nuclei, enhancing understanding of their spatial organization within complex biological systems.

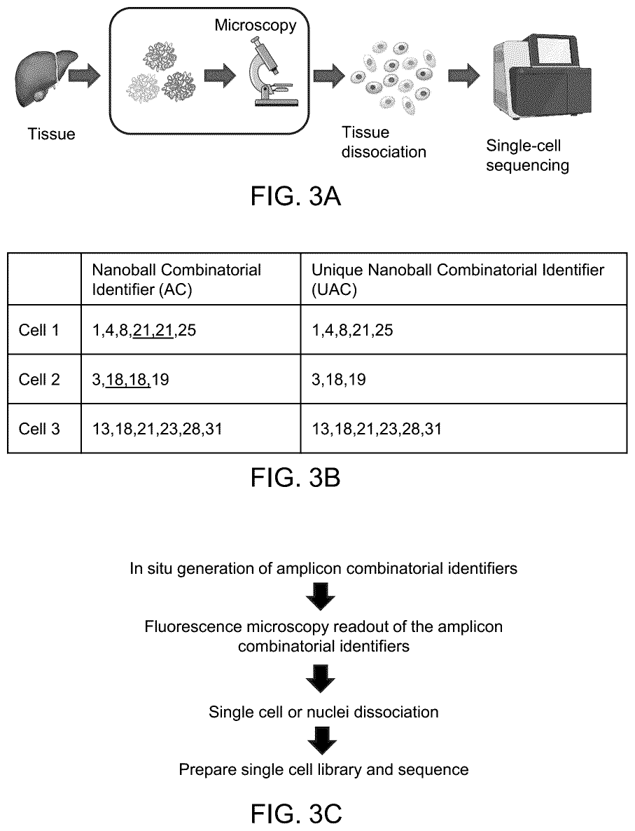

FIG. 3A-3C illustrates exemplary nanoball combinatorial indexing in accordance with one or more embodiment of the present invention.

In a preferred embodiment, both optical microscopy and next-generation sequencing (NGS) may allow comprehensive single-cell/nucleus analysis that combines the power of NGS with the spatial resolution of imaging techniques.

In accordance with an embodiment of the present invention, each cell may be indexed by its nanoball combinatorial identifier or unique nanoball combinatorial identifier.

The distribution of the nanoballs, among the cells or nuclei, may be random. The combination of the nanoballs creates a plurality of unique nanoball, for each of the cell or nucleus.

The combination of nanoballs creates a nanoball combinatorial identifier, while the combination of unique nanoballs creates a unique nanoball combinatorial identifier.

In an exemplary embodiment as shown in FIG. 3B, the cell 1 contains the nanoballs #1, 4, 8, 21, 21, and 25, while cell 2 contains the nanoballs #3, 18, 18, and 19. These cells can be discerned by their NCI. In some embodiments, the Unique Nanoball Combinatorial Identifiers (UNCIs) may be used, where each cell or nucleus may be assigned a combination of unique nanoballs.

In another exemplary embodiment, cell 1 has the unique nanoball #1, 4, 8, 21, and 25, while cell 2 has unique nanoball #3, 18, and 19, wherein only unique nanoballs are used. The number of possible combinations exponentially increases with the number of different nanoballs (n), allowing for a vast number of the cells or nuclei to be tagged in a tissue sample.

In some embodiments, the droplet-based approaches may include, 10×'s Chromium technique and the combination-based approaches may include split-pool technique. In a preferred embodiment, the integration of spatial and genomic data provides valuable insights into the cellular composition and organization within tissues.

In some embodiments, the fluorescence signal may be amplified using Tyramide Signal Amplification (TSA). The TSA may be a technique commonly employed to enhance the signal intensity in fluorescence-based assays. In an embodiment of the present disclosure, after the initial fluorescence staining of the nanoballs, a tyramide-labeled fluorophore may be applied, which undergoes enzymatic activation and generates multiple fluorophore molecules in close proximity to the target, resulting in signal amplification. This amplified fluorescence signal enhances the detection and visualization of the nanoballs during imaging.

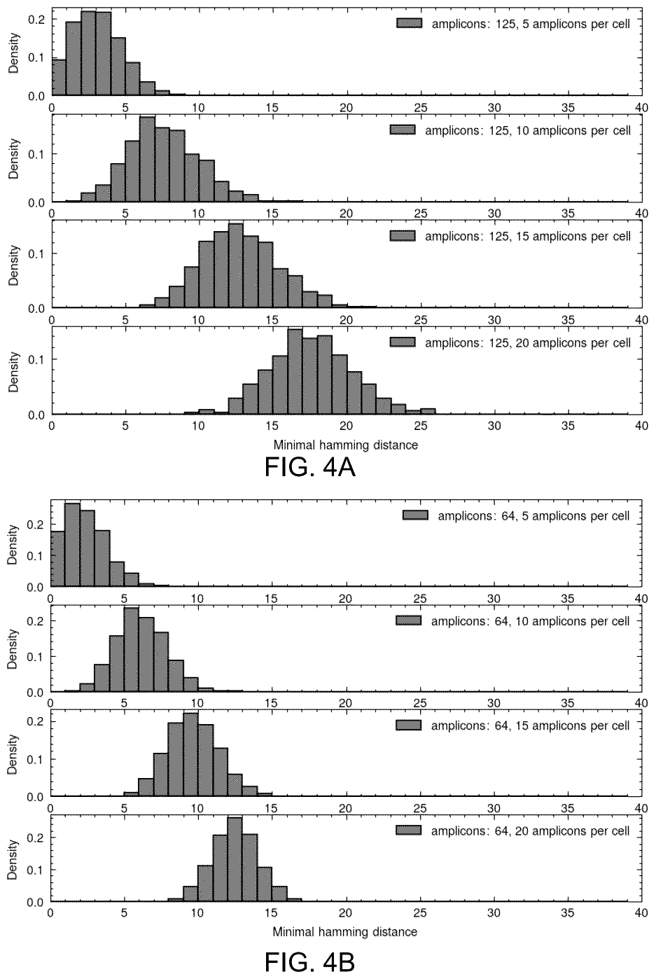

FIG. 4A-4B illustrates exemplary error correction in the nanoball combinatorial indexing in accordance with one or more embodiments of the present invention.

The Hamming distance, representing the number of differing unique nanoballs, may be used to measure the distance between two cells or nuclei. The histogram of the minimal Hamming distance for each of the cell or nucleus, indicating the distance to its nearest neighbor, may be plotted based on the average number of a unique nanoballs per cell/nucleus, which may be adjusted by the concentration of the stained oligos. A simulation demonstrates that when there are approximately 15 unique nanoballs per cell, the majority of cells or nuclei have a minimal Hamming distance may be greater than 5 from their nearest neighbor, providing ample redundancy for error correction and accurate cell or nucleus identification.

In a preferred embodiment, the capacity of Unique Nanoball Combinatorial Identifiers (UNCI) may be assessed through a Monte Carlo simulation as depicted in FIG. 4A and B. The simulation involves 100,000 cells and utilizes 125 and 64 different oligos, which are amplified into 125 and 64 unique nanoballs, respectively, as depicted in FIGS. 4A and B.

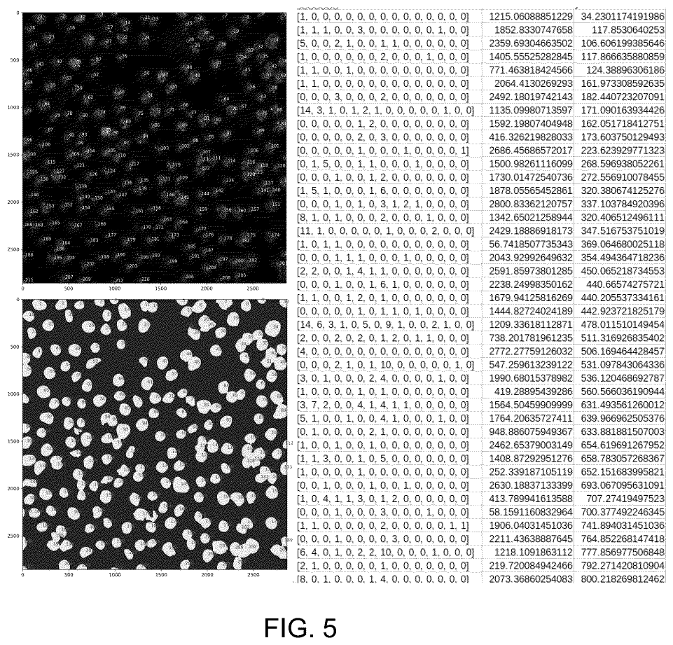

FIG. 5 illustrates a plurality of nanoball combinatorial identifiers index single cells in accordance with an embodiment of the present invention.

In a preferred embodiment, after imaging, masks are generated for each of the cells or nuclei. The combination of these nanoballs or the unique nanoballs provides the nanoball combinatorial identifier or unique nanoball combinatorial identifier, as shown in FIG. 5.

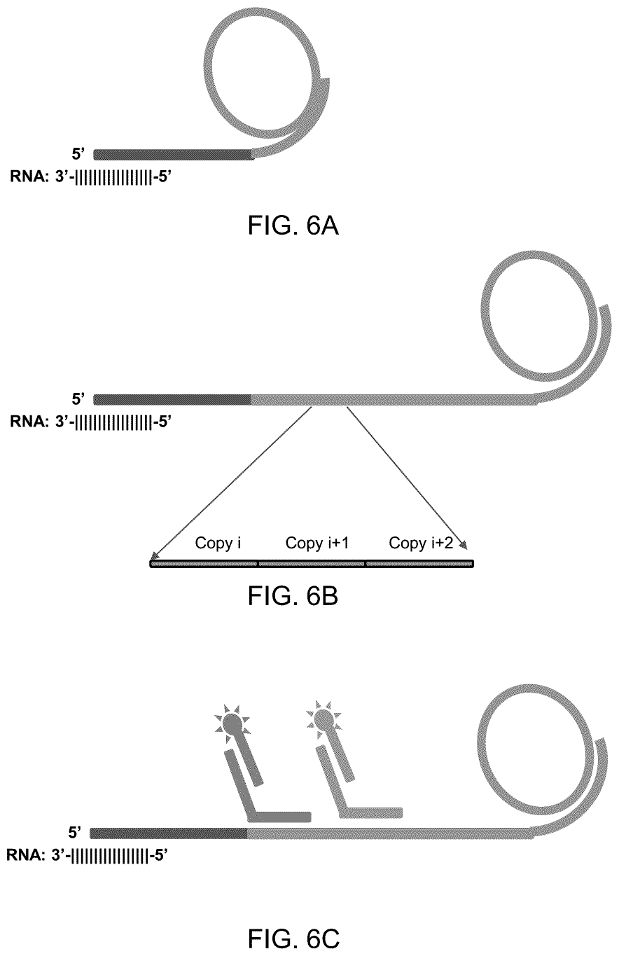

FIG. 6A-6C illustrates exemplary in situ nanoball generations in accordance with an embodiment of the present invention.

The single-cell or single-nucleus sequencing techniques may include droplet-based approaches or combination-based approaches.

The sequence of the nanoballs within each of the cell or nucleus may be determined using the respective single-cell sequencing.

In an exemplary embodiment (FIG. 6A), non-coding RNA Malat1 serves as an anchor for anchor oligos to bind and subsequently generate nanoballs. The anchors could be any DNA, RNA, or proteins within cells or nuclei. The anchor oligos may even non-specifically bind to the cells or nuclei.

A circular oligo may then be hybridized to the anchor oligo, and the anchor oligo extends via rolling circular amplification (RCA) to form a long single-stranded DNA with multiple repeats, which are nanoballs, as shown in FIG. 6B.

The nanoballs may be read out using multiplex fluorescence in situ hybridization (FISH) techniques, such as HyBiss, SeqFISH, RollFISH, or MERFISH. To enable readout, a set of bridge probes may be hybridized to the nanoball (FIG. 6C), and fluorescence dye-tagged FISH probes may subsequently be hybridized to the bridge probes, which may be imaged by a fluorescence microscope including, but not limited to, epi fluorescence, light-sheet, and confocal fluorescence microscopes. The readout may be achieved through multiple rounds of imaging, involving the stripping out, rehybridization of the bridge probes and FISH probes. If each round uses (k) colors, (m) rounds of imaging can encode k^{m} different unique nanoballs. For instance, 64 nanoballs may be read out through 3 rounds of 4-color imaging.

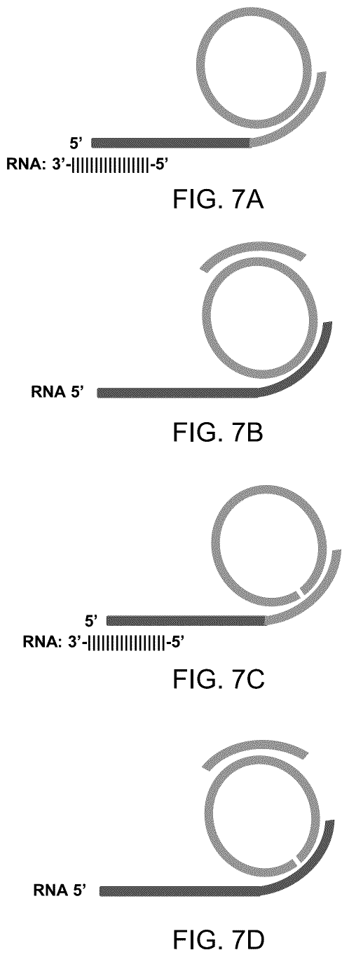

FIG. 7A-7D illustrates in-situ generation of the nanoball combinatorial identifiers in accordance with one or more embodiments of the present invention.

In an alternative embodiment to using an anchor oligo (shown in FIG. 7A), the circular oligo may directly hybridize to RNA, DNA, or oligo-tagged antibodies within the sample, as shown in FIG. 7B. This is followed by hybridization a DNA primer to the circular oligo for RCA extension.

In an exemplary embodiment, the generation of the circular oligo is achieved through in situ ligation of a linear DNA oligo, where the 5′ and 3′ ends of the linear oligo may be hybridized to the anchor oligo, as shown in FIG. 7C. The linear oligo may commonly be known as a padlock oligo. Additionally, the circular oligo may be generated from a linear oligo hybridized to an RNA molecule, as shown in FIG. 7D. A DNA primer may then be hybridized to the circular probe for RCA extension. The proposed method provides flexibility in generating the circular oligo for subsequent RCA amplification.

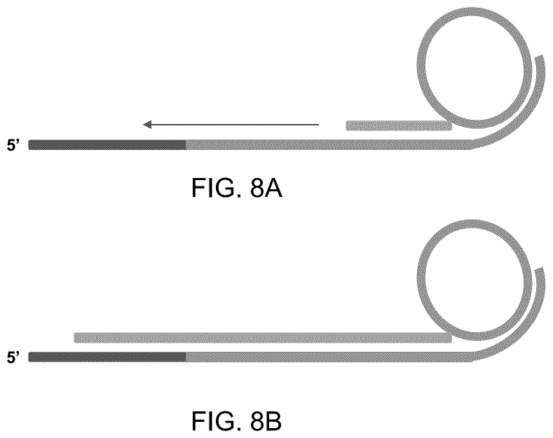

FIG. 8A-8B illustrates a readout of the nanoball combinatorial identifiers via sc-ATAC-seq in accordance with one or more embodiments of the present invention.

The sequencing of the nanoballs may be performed using a single-cell ATAC-seq process which may include hybridization of a primer to the nanoball after optical imaging, extending the hybridized nanoball through a polymerase to create a double-stranded DNA, and sequencing double-stranded DNA by following single-cell ATAC-seq protocols, including but not limited to sc-ATAC-seq and 10×'s sc-ATAC-seq kit.

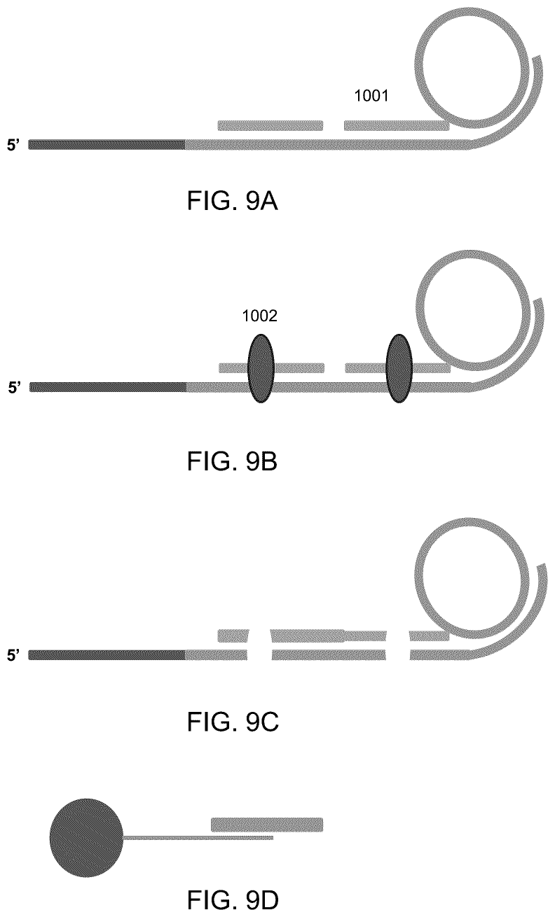

FIG. 9A-9D illustrates a plurality of the nanoball combinatorial identifiers captured by beads with sc-ATAC-seq or sc-RNA-seq in accordance with one or more embodiments of the present invention.

The sequencing of the nanoballs may be performed using a 10×'s Chromium single-cell RNA sequencing kit when the nanoballs contain sequences of endonuclease restriction sites. The process of using the 10×'s Chromium single-cell RNA sequencing kit may include hybridizing oligos after optical imaging to the endonuclease restriction sites, fragmenting the nanoballs using endonucleases to release capture sequences or fragments, and allowing the fragments to be captured by the beads provided by the Chromium sc-RNA-seq kit.

In an embodiment of the present disclosure, after optical imaging, oligos may hybridize to endonuclease restriction sites, as shown in FIG. 9A. Endonucleases may then be used to fragment the nanoballs, as shown in FIGS. 9B and C. The nanoballs may also contain capture sequences that allows their fragments to be captured by the beads provided by the Chromium sc-RNA-seq kit, as shown in FIG. 9D. Hence, sequencing the captured fragments can be accomplished using the 10×'s Chromium single-cell RNA sequencing protocol.

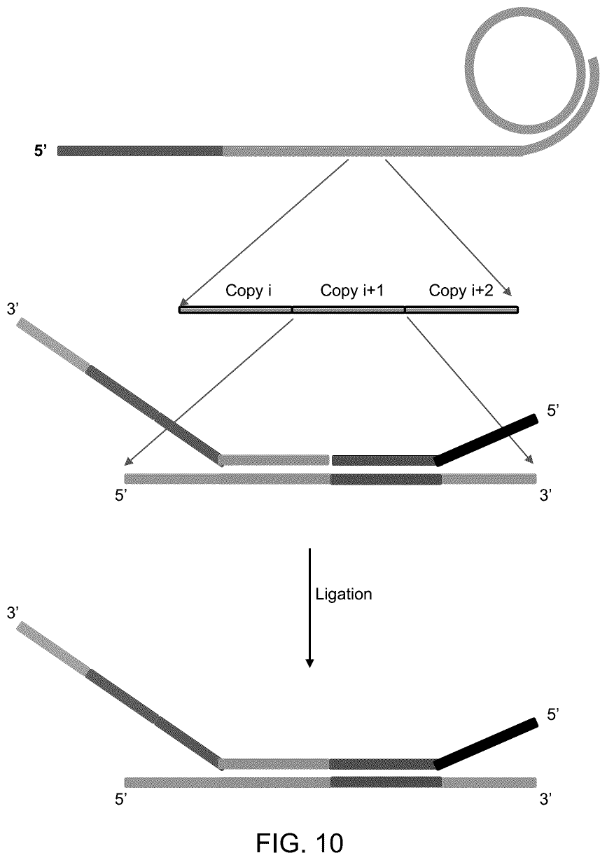

FIG. 10 illustrates a readout of the nanoball combinatorial identifiers using sc-RNA-seq in accordance with an embodiment of the present invention.

The sequencing of the nanoballs may be performed using a 10×'s Chromium fixed RNA protocol. The protocol includes hybridizing a pair of DNA oligos to the nanoballs and subsequently ligating them together, allowing the ligated oligos to be sequenced following the 10×'s Chromium fixed RNA protocol.

In an embodiment of the present disclosure, the remaining sequence of the oligo pairs may be the same as the 10×'s oligoes, which allows the ligated oligos to be sequenced following the 10×'s Chromium fixed RNA protocol.

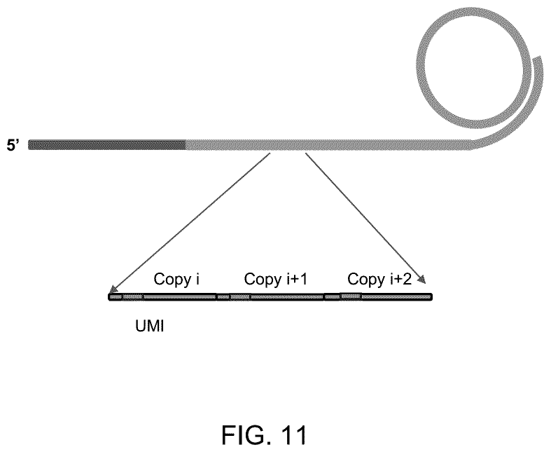

FIG. 11 illustrates a unique molecular identifier (UMI) for counting the nanoballs in accordance with an embodiment of the present invention.

The nanoballs may include a unique molecular identifier (UMI).

The unique molecular identifier (UMI) may serve as a molecular tag that enables accurate counting of the non-unique nanoballs during sequencing.

The method 200 may further comprise incorporating unique molecular identifier, during counting, a plurality of identical nanoballs may be distinguished and counted as separate entities that allows for correlation between the microscopy count and the sequencing count of the non-unique nanoballs.

In an exemplary embodiment, if cell 1 contains two nanoballs of nanoball #21, microscopy would detect these two nanoballs. The presence of different UMIs ensures that the sequencing step can accurately count two nanoballs of the nanoball #21 as well, as depicted in FIG. 3B.

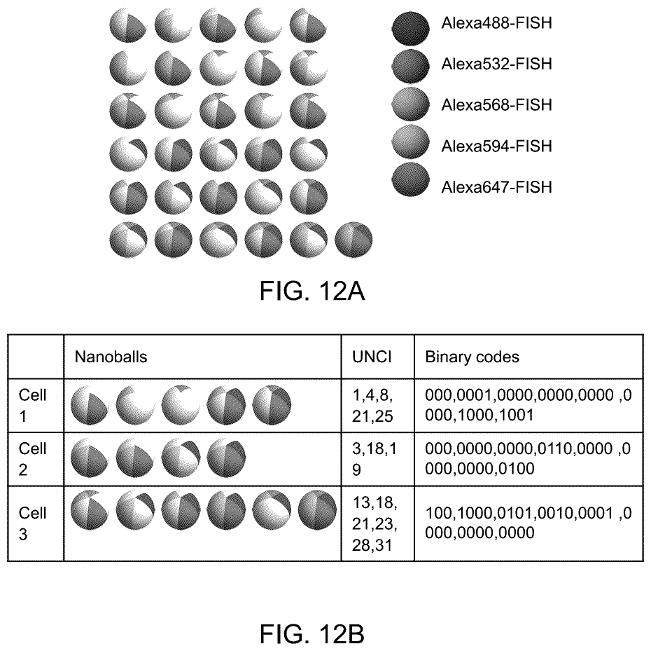

FIG. 12A-12B illustrates an exemplary color combinatorial coding in accordance with one or more embodiments of the present invention.

The imaging of the nanoballs may be performed by employing a color combinatorial coding approach.

In an embodiment of the present disclosure, each of the nanoball may be imaged using multiple colors in the same round, employing a color combinatorial coding approach. This allows for a reduction in the number of imaging rounds required. In an exemplary embodiment, when utilizing five different colors, the nanoball #1 may be hybridized to the Alexa647-FISH probe exclusively, the nanoball #2 to the Alexa594-FISH probe exclusively, and the nanoball #3 to both the Alexa647 and Alexa594-FISH probes. The nanoball #31 can be hybridized to the Alexa647, Alexa594, Alexa568, Alexa532, and Alexa488-FISH probes simultaneously. With this approach, five colors can encode 31 unique nanoballs in a single imaging round, effectively reducing the overall imaging time and complexity, as depicted in FIG. 12A. These combinations of the nanoballs may then be served to index the individual cells or nuclei within the sample.

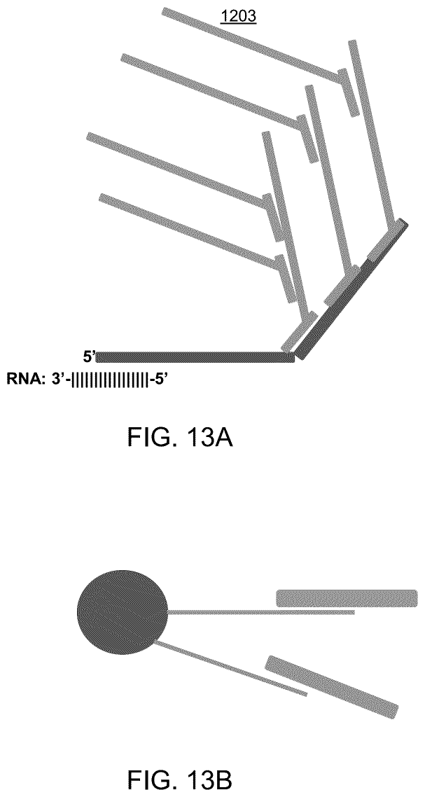

FIG. 13A-13B illustrates an in-situ generation of the nanoball combinatorial identifier hybridization in accordance with one or more embodiments of the present invention.

The nanoballs may be generated in situ using multiple rounds of hybridization.

The multiple rounds of hybridization for generating the nanoballs may further include staining a first set of oligos with nucleic acid or oligo-tagged antibodies in a sample; hybridizing a second set of oligos to the first plurality, with the second set being specific to the first set; subsequently hybridizing a third set of oligos to the second plurality; and reiterating the process for a predetermined number of iterations.

In a preferred embodiment, the nanoballs may be generated in situ using multiple rounds of hybridization which may be similar to hybridization-based amplification methods used in HCR (Choi et al. 2010), RNAscope (Wang et al. 2012), Immuo-Sabre (Saka et al. 2019), SeqFISH (Cai et al. 2014) (Eng et al. 2019), MERFISH (Zhuang et al. 2015) (Moffitt and Zhuang 2016), or Nanostring's CoxMX-SMI (He et al. 2022). In one embodiment, a plurality of oligos is first stained with nucleic acid or oligo-tagged antibodies in a sample, as depicted in FIG. 13A. A second plurality of oligos may then be hybridized to the first plurality, with the second plurality being specific to the first plurality, as depicted in FIG. 13A. Subsequently, a third plurality of oligos may be specifically hybridized to the second plurality, as depicted in FIG. 13A. After multiple rounds of hybridizations, the oligos from different rounds may cluster together spatially, forming the nanoballs. The nanoballs may be optically read out using fluorescence in situ hybridization (FISH) techniques. Following microscopy readout, the oligos can be captured by beads for single-cell sequencing, as depicted in FIG. 13B. FIG. 13B also depicts the capture of the hybridization-generated nanoballs by beads provided in the sc-RNA-seq kit for subsequent readout of the nanoball combinatorial identifier.

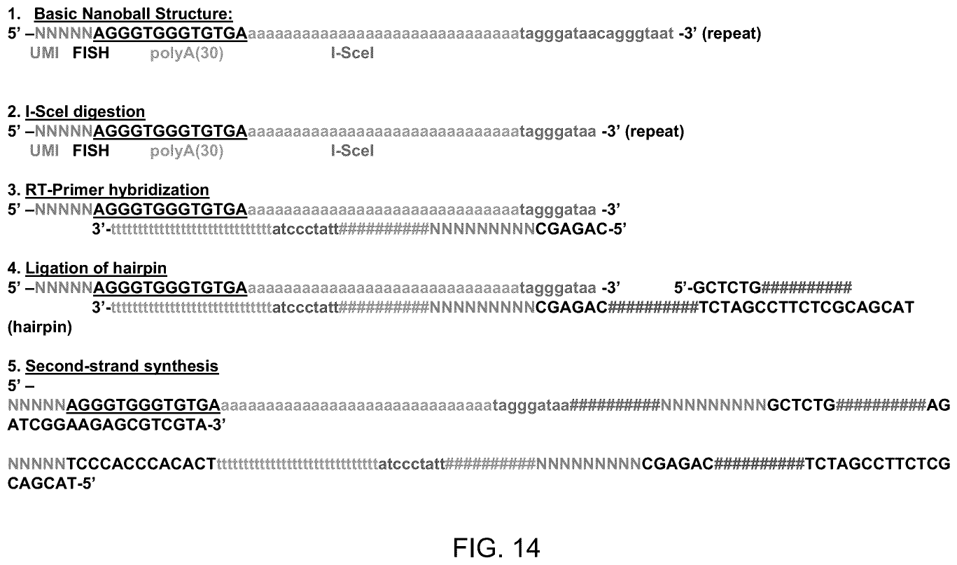

FIG. 14 illustrates a readout of the nanoball combinatorial identifiers via sci-RNA-seq in accordance with an embodiment of the present invention.

The nanoballs may be read out using sci-RNA-seq kit when the nanoballs include restriction sites. This process involves hybridizing the nanoballs containing a polyA sequence to a reverse-transcription primer (RT-primer), using a hairpin oligo to ligate to the reverse-transcription primer (RT-primer), performing elongation using the reverse-transcription primer (RT-primer) as a template, and incorporating the sci-RNA-seq barcodes to enable the generation of the sci-RNA-seq library for the nanoball readout.

In some embodiments, the nanoball includes a restriction site, including but not limited to, I-SceI, which can be utilized to fragment the single-strand RCA product into shorter segments. Following hybridization to the RT primer, a hairpin oligo from the sci-RNA-seq kit can hybridize and ligate to the RT primer. During the second-strand synthesis step of the single cell combinatorial indexing RNA sequencing (sci-RNA-seq) (Martin et al. 2023; Srivatsan et al. 2021) protocol, the fragmented RCA product is elongated using the RT primer as a template, thereby incorporating the single cell combinatorial indexing RNA sequencing (sci-RNA-seq) barcodes and enabling the generation of the single cell combinatorial indexing RNA sequencing (sci-RNA-seq) library.

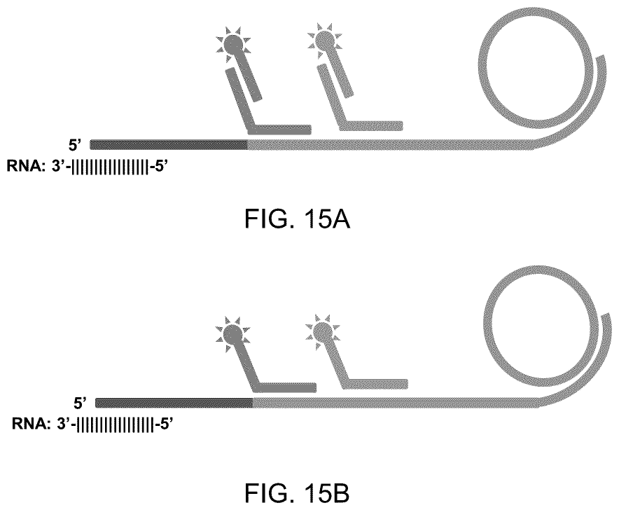

FIG. 15A-15B illustrates a fluorescence microscopy readout in accordance with one or more embodiments of the present invention.

In an embodiment of the present disclosure, the FISH probes tagged with fluorophores may directly hybridize to the nanoball (FIG. 15B). This eliminates the need for the hybridization and rehybridization of bridge oligos (FIG. 15A), simplifying the readout process.

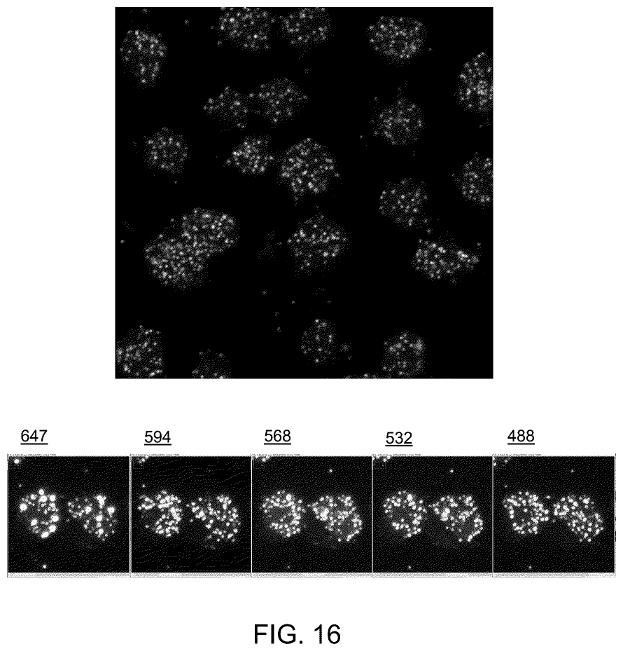

FIG. 16 illustrates a plurality of the nanoball combinatorial identifiers in cells in accordance with an embodiment of the present invention.

In an embodiment of the present disclosure, the number of fluorophores used may not be limited to four, as demonstrated in FIG. 16. In an embodiment, a 5-color system may be employed, utilizing CF647, CF594, CF568, CF532, and CF488 dyes. CF-dyes are purchased from Biotium. There are a large quantity of choices of fluorescence dyes that can be used and are not limited to CF-series or Alexa series. In an alternate embodiment, the incorporation of machine learning algorithms may extend the color palette to include 15 or more colors.

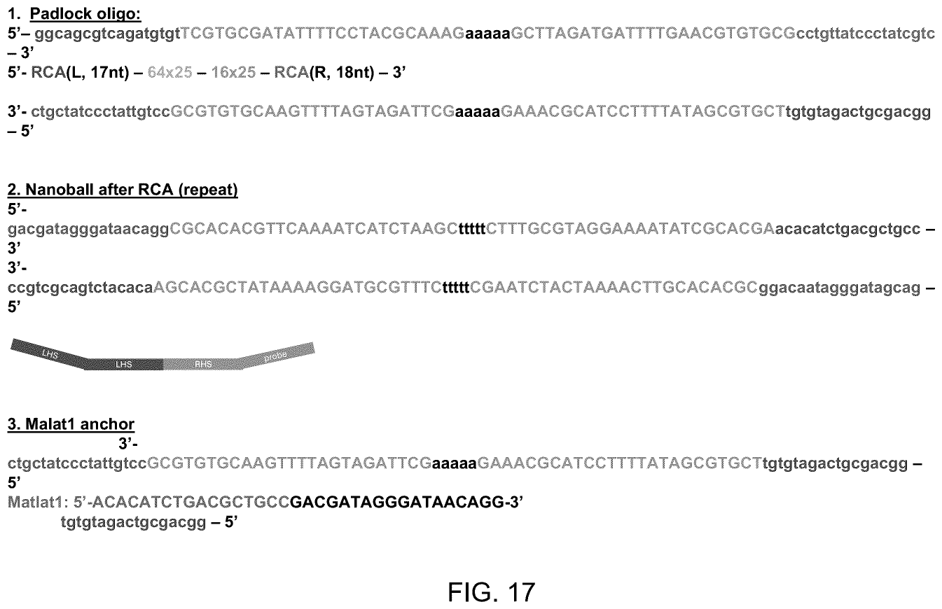

FIG. 17 illustrates a readout of an exemplary nanoball in accordance with an embodiment of the present invention.

In an exemplary embodiment, the nanoball as illustrated in FIG. 17. The nanoball consists of a padlock oligo with four segments: RCA left arm, bridge segment 1, bridge segment 2, and RCA right arm. The padlock oligo can hybridize to the 3′ tail of a Malat1 probe, enabling ligation to form a circular oligo. Phosphorylation of the 5′ end of the padlock oligo enhances ligation efficiency. There are 64 different padlock oligos, which are amplified into 64 nanoballs after RCA. Optical readout of the nanoballs is achieved through three rounds of four-color imaging. The two bridge segments are utilized for hybridization during the three rounds of FISH imaging. The first segment contains 64 different sequences, while the second segment has 16 different sequences. In each imaging round, specific bridge probes hybridize to their respective segments Subsequently, the bridge probes hybridize to the four-color FISH probes. After imaging, a pair of oligos, referred to as LHS and RHS, hybridize to the nanoball and are ligated together. The RHS probe is specific to segment 1, which contains 64 different unique sequences, while the LHS probe is specific to the left anchor region of RCA and remains constant for all 64 nanoballs. Following dissociation of the cells or nuclei, sequencing is performed using the standard sc-RNA-seq protocol.

In an exemplified embodiment, a sample consisting of cells is cytospun onto a glass slide. The cells are first fixed with 2% PFA (paraformaldehyde) in PBS (phosphate buffer saline) for 10 minutes. After washing with PBS three times for 5 minutes each, the cells are permeabilized in a 0.1% Triton X-100 in PBS solution for 10 minutes. The sample is washed again three times for 5 minutes each in PBS. Next, the sample is hybridized at 37° C. overnight with Malat1 probe at a concentration of 30 nM in a hybridization buffer. The hybridization buffer includes 20% formamide in 2×SSC (saline-sodium citrate) buffer. After hybridization, the sample is washed three times for 5 minutes each in a 20% formamide in 2×SSC buffer. A series of blank probes without a 3′ tail are mixed with the Malat1 probes to enable competitive binding and controlling the concentration of the nanoballs per cell. The ratio between the blank and non-blank could be 1:1 to 1:10, depending on the quality of the samples. Padlock probes (Nilsson et al. 1994) with concentrations ranging from 30 nM to 100 nM are then hybridized overnight to the Malat1 probes. After washing once in a 20% formamide and 2×SSC buffer at 37° C., T4 ligase is used to ligate the padlock probes into circular probes at room temperature for approximately 3 hours.

Following ligation, the phi29 enzyme from NEB (New England Biolabs) is used for Rolling Circle Amplification (RCA) (Church et al. 2015, Glezer et al. 2021) (Mohsen and Kool 2016) overnight at 37° C. in a water bath. The sample is then washed three times for 5 minutes each in a 20% formamide and 2×SSC buffer. Prior to imaging, the sample is hybridized with bridge probes (Gyllborg et al. 2020) at a concentration of 30 nM in a 20% formamide and 2×SSC buffer for 3 hours at 37° C. This is followed by three-time washing with 20% formamide in 2×SSC buffer for 5 minutes each at 37° C. FISH probes are then hybridized to the sample in the same 20% formamide and 2×SSC buffer for 3 hours (Gyllborg et al. 2020). The sample is washed again with 20% formamide in 2×SSC buffer for three times at 37° C. Finally, the sample is mounted using Prolong Gold or Vector-Shield antifading medium. A Zeiss LSM confocal microscope is used for four-color imaging. The images are taken at multiple Z-sections with a separation of 0.5 micrometers per slice. A 20×0.75NA lens is utilized. After the first round of imaging, the sample is incubated in PBS buffer at 37° C. for 1 hour to remove the cover glass. The bridge and FISH probes are then stripped using 65% formamide in 2×SSC for 30 minutes. The sample is stained with the second round of bridge probes and FISH probes, following the same protocol as the first round. This process is repeated for three rounds to read out all 64 nanoballs. The images from the three rounds are aligned using mutual information or cross-correlation algorithms in Python (Foroosh, Zerubia, and Berthod 2002). The 3D images are then maximal projected into 2D. The nanoballs are detected using ThunderSTORM (Ovesny et al. 2014), a common single-molecule localization microscopy software.

A neural network model is further employed to verify the correct detections. CellPose (Pachitariu and Stringer 2022; Stringer et al. 2021) is used to segment the cells or nuclei. The unique nanoballs within each of the cell or nucleus are utilized to generate the unique nanoball combinatorial identifier of the cells. After imaging and stripping of the bridge and FISH probes, LHS and RHS probes, both at a concentration of 30 nM, are hybridized to the sample. They are then ligated using T4 ligase for 3 hours. The remaining oligos are washed with 20% formamide in 2×SSC buffer. The samples are then used for single-cell RNA sequencing (sc-RNA-seq) following the 10×'s Chromium fixed RNA sequencing protocol (Janesick et al. 2022).

In some embodiments, for oligo or oligonucleotide amplification, short DNA sequences (oligonucleotides) may be synthesized using standard chemical methods. In some embodiments, oligo may be amplified using PCR, which involves repeatedly heating and cooling the DNA to denature it, anneal primers, and extend the DNA strands. This results in a large number of copies of the original DNA sequence.

In some embodiments, the circularization of amplified DNA may be performed using adaptor ligation, where the amplified DNA fragments may be ligated to adaptors, which are short double-stranded DNA sequences that facilitate subsequent processes. In some embodiments, the DNA fragments are circularized to form single-stranded DNA circles performed by intramolecular ligation, where the ends of the DNA fragment are joined to form a circle.

In some embodiments, DNA nanoballs are formed when long single-stranded DNA generated by Rolling Circle Amplification (RCA) folds upon itself, creating compact and dense structures known as DNA nanoballs. These nanoballs are stabilized through molecular interactions, ensuring that they maintain their compact structure. Each of the nanoball contains numerous copies of the original DNA sequence, enhancing the signal for sequencing.

In some embodiments, for hybridization, various oligonucleotide probes are used that can hybridize to target sequences within the cells or nuclei. These probes often have sequences complementary to the target DNA or RNA. This may involve in situ hybridization techniques where fluorescently labeled probes bind to their complementary sequences within the sample. In some embodiments, after hybridization, target sequences or probes are circularized if they aren't already in circular form. The circularized DNA is then amplified, producing long single-stranded DNA molecules that fold into compact the DNA nanoballs. These DNA nanoballs contain multiple copies of the target sequence, enhancing the signal for detection.

In some embodiments, During RCA or subsequent processing, fluorescent labels are incorporated in the nanoballs to facilitate optical detection under a microscope. In some embodiments, a high-resolution fluorescence microscope equipped with the appropriate filters is used to detect the fluorescent labels, which involves acquiring images of the sample and capturing the fluorescence emitted by the nanoballs. Each of the nanoball may appear as a bright spot in the image.

In some embodiments, for spatial coordinate recording, image analysis software is used to detect and record the positions of each fluorescent spot within the sample. This involves identifying the coordinates (x, y, and sometimes z) of each spot in the image. In some embodiments, to correlate the spatial coordinates of the nanoballs with the underlying cellular or tissue structures, overlaying the fluorescence images with bright-field images is used to show the cellular context.

In some embodiments, integrating the spatial coordinates with sequencing data allows the mapping of sequences to precise locations within the tissue or cells. In some embodiments, bioinformatics analysis to interpret the spatial distribution of genetic or transcriptomic information can reveal insights into the spatial organization of gene expression, genetic mutations, or other molecular features within the sample.

In some embodiments, the tissue dissociation is performed by mechanical dissociation or physically mincing the tissue using scalpels, blades, or tissue grinders (Yadav et al. 2022; Vallejo et al. 2022; Chung et al. 2022). In some embodiments, tissue dissociation is performed through enzymatic digestion using enzymes such as collagenase, trypsin, or dispase to break down the extracellular matrix and release individual cells (Yadav et al. 2022; Chung et al. 2022). The specific enzymes or combination of enzymes depend on the tissue type.

In some embodiments, the tissue dissociation is performed by mincing the tissue into small pieces, incubating the tissue pieces in an enzyme solution at an appropriate temperature (e.g. 37° C.) with gentle agitation, monitoring the dissociation progress under a microscope, neutralizing the enzymes using a suitable inhibitor (e.g., fetal bovine serum). In some embodiments, after dissociation, the cells or nuclei suspension may be washed and filtered to remove debris and undigested tissue fragments, followed by gently centrifugating to pellet the cells or nuclei, resuspending the cells or nuclei in a fresh buffer (e.g., PBS with 0.04% BSA), repeating the centrifugation, and passing the cell suspension through a cell strainer (e.g. 40-70 μm) to remove clumps and obtain a uniform single-cell suspension.

In some embodiments, the sequencing data may be analyzed to reveal the transcriptomic profiles of individual cells through various techniques such as, barcode demultiplexing, read alignment, quantification, clustering, differential expression analysis, and visualization to interpret the single-cell data.

In a case that no conflict occurs, the embodiments in the present disclosure and the features in the embodiments may be mutually combined. The foregoing descriptions are merely specific implementations of the present disclosure, but are not intended to limit the protection scope of the present disclosure. Any variation or replacement readily figured out by a person skilled in the art within the technical scope disclosed in the present disclosure shall fall within the protection scope of the present disclosure. Therefore, the protection scope of the present disclosure shall be subject to the protection scope of the claims.

The foregoing descriptions of specific embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

### REFERENCE CITED

Choi, Harry MT, Joann Y Chang, Le A Trinh, Jennifer E Padilla, Scott E Fraser, and Niles A Pierce. 2010. ‘Programmable in situ amplification for multiplexed imaging of mRNA expression’, Nature Biotechnology, 28: 1208-12.
Chung, Hattie, Alexandre Melnikov, Cristin McCabe, Eugene Drokhlyansky, Nicholas Van Wittenberghe, Emma M Magee, Julia Waldman, Avrum Spira, Fei Chen, and Sarah Mazzilli. 2022. ‘SnFFPE-Seq: towards scalable single nucleus RNA-Seq of formalin-fixed paraffin-embedded (FFPE) tissue’, bioRxiv: 2022.08. 25.505257.
Eng, C. L., M. Lawson, Q. Zhu, R. Dries, N. Koulena, Y. Takei, J. Yun, C. Cronin, C. Karp, G. C. Yuan, and L. Cai. 2019. ‘Transcriptome-scale super-resolved imaging in tissues by RNA seqFISH’, Nature, 568:235-39.
Foroosh, Hassan, Josiane B Zerubia, and Marc Berthod. 2002. ‘Extension of phase correlation to subpixel registration’, IEEE transactions on image processing, 11: 188-200.
Gyllborg, D., C. M. Langseth, X. Qian, E. Choi, S. M. Salas, M. M. Hilscher, E. S. Lein, and M. Nilsson. 2020. ‘Hybridization-based in situ sequencing (HybISS) for spatially resolved transcriptomics in human and mouse brain tissue’, Nucleic Acids Res, 48: e112.
He, Shanshan, Ruchir Bhatt, Carl Brown, Emily A Brown, Derek L Buhr, Kan Chantranuvatana, Patrick Danaher, Dwayne Dunaway, Ryan G Garrison, and Gary Geiss. 2022. ‘High-plex imaging of RNA and proteins at subcellular resolution in fixed tissue by spatial molecular imaging’, Nature Biotechnology, 40: 1794-806.
Janesick, Amanda, Robert Shelansky, Andrew D. Gottscho, Florian Wagner, Morgane Rouault, Ghezal Beliakoff, Michelli Faria de Oliveira, Andrew Kohlway, Jawad Abousoud, Carolyn A. Morrison, Tingsheng Yu Drennon, Seayar H. Mohabbat, Stephen R. Williams, and Sarah E. B. Taylor. 2022. ‘High resolution mapping of the breast cancer tumor microenvironment using integrated single cell, spatial and in situ analysis of FFPE tissue’, bioRxiv: 2022.10.06.510405.
Martin, B. K., C. Qiu, E. Nichols, M. Phung, R. Green-Gladden, S. Srivatsan, R. Blecher-Gonen, B. J. Beliveau, C. Trapnell, J. Cao, and J. Shendure. 2023. ‘Optimized single-nucleus transcriptional profiling by combinatorial indexing’, Nat Protoc, 18: 188-207.
Moffitt, J. R., and X. Zhuang. 2016. ‘RNA Imaging with Multiplexed Error-Robust Fluorescence In Situ Hybridization (MERFISH)’, Methods Enzymol, 572: 1-49.
Mohsen, M. G., and E. T. Kool. 2016. ‘The Discovery of Rolling Circle Amplification and Rolling Circle Transcription’, Acc Chem Res, 49: 2540-50.
Nilsson, M., H. Malmgren, M. Samiotaki, M. Kwiatkowski, B. P. Chowdhary, and U. Landegren. 1994. ‘Padlock probes: circularizing oligonucleotides for localized DNA detection’, Science, 265: 2085-8.
Ovesny, M., P. Krizek, J. Borkovec, Z. Svindrych, and G. M. Hagen. 2014. ‘ThunderSTORM: a comprehensive ImageJ plug-in for PALM and STORM data analysis and super-resolution imaging’, Bioinformatics, 30: 2389-90.
Pachitariu, Marius, and Carsen Stringer. 2022. ‘Cellpose 2.0: how to train your own model’, Nature Methods, 19: 1634-41.
Saka, Sinem K, Yu Wang, Jocelyn Y Kishi, Allen Zhu, Yitian Zeng, Wenxin Xie, Koray Kirli, Clarence Yapp, Marcelo Cicconet, and Brian J Beliveau. 2019. ‘Immuno-SABER enables highly multiplexed and amplified protein imaging in tissues’, Nature Biotechnology, 37: 1080-90.
Srivatsan, S. R., M. C. Regier, E. Barkan, J. M. Franks, J. S. Packer, P. Grosjean, M. Duran, S. Saxton, J. J. Ladd, M. Spielmann, C. Lois, P. D. Lampe, J. Shendure, K. R. Stevens, and C. Trapnell. 2021. ‘Embryo-scale, single-cell spatial transcriptomics’, Science, 373: 111-17.
Stringer, C., T. Wang, M. Michaelos, and M. Pachitariu. 2021. ‘Cellpose: a generalist algorithm for cellular segmentation’, Nat Methods, 18: 100-06.
Vallejo, Andres F, Kate Harvey, Taopeng Wang, Kellie Wise, Lisa M Butler, Jose Polo, Jasmine Plummer, Alex Swarbrick, and Luciano G Martelotto. 2022. ‘snPATHO-seq: unlocking the FFPE archives for single nucleus RNA profiling’, bioRxiv. 2022.08. 23.505054.
Wang, F., J. Flanagan, N. Su, L. C. Wang, S. Bui, A. Nielson, X. Wu, H. T. Vo, X. J. Ma, and Y. Luo. 2012. ‘RNAscope: a novel in situ RNA analysis platform for formalin-fixed, paraffin-embedded tissues’, J Mol Diagn, 14: 22-9.
Yadav, Ram Prakash, Vamsi Krishna Polavarapu, Pengwei Xing, and Xingqi Chen. 2022. ‘FFPE-ATAC: A Highly Sensitive Method for Profiling Chromatin Accessibility in Formalin-Fixed Paraffin-Embedded Samples’, Current protocols, 2: e535.
Cai L, Lubeck E, Zhiyentayev T, Coskun A, He T-F, Sohn CH, Shah S, inventors; California Institute of Technology, assignee. Multiplex labeling of molecules by sequential hybridization barcoding patent US 20150267251 A1. 2014.
Church GM, Lee J-H, Daugharthy ER, inventors; Harvard College, assignee. Methods for high-throughput labelling and detection of biological features in situ using microscopy patent US 20230227895 A1. 2015.
Zhuang X, Chen K-H, Boettiger A, Moffitt JR, Wang S, inventors; Harvard College, assignee. Systems and methods for determining nucleic acids. U.S. Ser No. 11/098,303B2. 2015.
Glezer EN, Cang H, Hong Z, inventors; Singular Genomics Systems Inc, assignee. Methods for in situ transcriptomcis and proteomics patent U.S. Ser. No. 11/492,662B2. 2021.

What is claimed for:

1. A method for spatial single-cell sequencing, the method comprising:
collecting a sample comprising a plurality of cells or nuclei, wherein each of the cells or nuclei contains multiple oligos;
amplifying oligos and generating a plurality of nanoballs within each of the cells or nuclei;
creating a nanoball combinatorial identifier (NCI) or a unique nanoball combinatorial identifier (UNCI) for each of the cells or nuclei based on the combination of nanoballs, wherein the combination of the nanoballs within each of the cells or nuclei create the nanoball combinatorial identifier (NCI) and the combination of the unique nanoballs serves as the unique nanoball combinatorial identifier (UNCI);
identifying the nanoballs using both optical microscopy and next-generation sequencing (NGS)-based single-cell sequencing assays;
dissociating the cells or nuclei from tissues, and subjecting them to single-cell or single-nucleus sequencing;
indexing the spatial information of each of the cells or nuclei using the combinations of the nanoballs; and
subsequently sequencing the nanoballs to correlate the spatial location/coordinates of each cell or nucleus with the single-cell sequencing data, wherein the single-cell sequencing data includes single-cell-RNA-sequencing (sc-RNA-seq) data, single-cell assay for transposase-accessible chromatin using sequencing (sc-ATAC-seq) data, single-cell chromatin immunoprecipitation sequencing (sc-ChIP-seq) data, single-cell (sc)-Methylation-seq data, single-cell (sc)-clonal sequencing data.

2. The method as claimed in claim 1, wherein during the nanoball generation unique molecular identifiers (UMIs) are used to distinguish non-unique nanoballs.

3. The method as claimed in claim 1, wherein the nanoball combinatorial identifier (NCI) or the unique nanoball combinatorial identifier (UNCI) is detectable by both optical microscopy and single-cell or single-nucleus sequencing, enabling the correlation of spatial coordinates of the tagged cells or nuclei with single-cell or single-nuclei sequencing data, including single-cell-RNA-sequencing (sc-RNA-seq), single-cell assay for transposase-accessible chromatin using sequencing (sc-ATAC-seq), single-cell chromatin immunoprecipitation sequencing (sc-ChIP-seq), single-cell (sc)-Methylation-seq, and single-cell (sc)-clonal seq.

4. The method as claimed in claim 1, wherein the nanoballs are generated by rolling circular amplification (RCA) through a plurality of circularized linear oligo or a plurality of padlock oligo anchored to the cells or nuclei, and subsequently circularized via ligation.

5. A method for enabling the spatial tagging of the single cells or nuclei within tissues, the method comprising:
collecting a plurality of biological samples such as, tissues and cells and performing fixation and permeabilization on the collected biological samples;
employing a set of deoxyribonucleic acid (DNA) oligos to stain the cells or nuclei within the tissues;
amplifying each deoxyribonucleic acid (DNA) oligo to generate a plurality of replica located together as the nanoballs;
employing optical microscopy to perform imaging and read out the nanoballs and enabling the recording of spatial coordinates for each of the cells or nuclei;
segmenting the cells or nuclei using a plurality of algorithms including, but not limited to Otsu, watershed, and neural networks, to generate masks for each of the cells or nuclei;
using the masks to identify the nanoballs within each of the cells or nuclei;
subsequently dissociating the cells or nuclei for single-cell or single-nucleus isolation;
subjecting the isolated single cells or nuclei to a plurality of single-cell or single-nucleus sequencing techniques;
determining the sequence of the nanoballs within each of the cells or nuclei and enabling the correlation of genomic information with spatial coordinates; and
integrating the obtained spatial and genomic data to gain valuable insights into the cellular composition and organization within tissues.

6. The method as claimed in claim 5, wherein the distribution of the nanoballs, among the cells or nuclei is random and the combination of the nanoballs creates the identifiers for each cell or nucleus.

7. The method as claimed in claim 6, wherein the combination of the nanoballs and the unique nanoballs creates the nanoball combinatorial identifier or the unique nanoball combinatorial identifier, respectively.

8. The method as claimed in claim 5, wherein the method further comprises dissociating the cells or nuclei using a plurality of enzymatic or mechanical processes.

9. The method as claimed in claim 5, wherein the single-cell or single-nucleus sequencing techniques include droplet-based approaches or combination-based approaches or hydrogel-based approaches or microfluidic-based approaches.

10. The method as claimed in claim 5, wherein the sequence of the nanoballs within each of the cells or nuclei is determined using the respective single-cell sequencing.

11. The method of claim 5, wherein the sequencing of the nanoballs is performed using a single-cell assay for transposase-accessible chromatin using sequencing (sc-ATAC-seq) process, and the single-cell assay for transposase-accessible chromatin using sequencing (sc-ATAC-seq) process includes:
hybridization of a primer to the nanoball after optical imaging;
extending the hybridized nanoball through a polymerase to create a double-stranded deoxyribonucleic acid (DNA); and
sequencing double-stranded deoxyribonucleic acid (DNA) by following single-cell assay for transposase-accessible chromatin using sequencing (sc-ATAC-seq), including but not limited to single-cell assay for transposase-accessible chromatin using sequencing (sc-ATAC-seq) and 10×'s single-cell assay for transposase-accessible chromatin using sequencing (sc-ATAC-seq).

12. The method as claimed in claim 5, wherein the sequencing of the nanoballs is performed using a 10×'s Chromium single-cell ribonucleic acid (RNA) sequencing kit when the nanoballs contain sequences of endonuclease restriction sites and the process of using the 10×'s Chromium single-cell ribonucleic acid (RNA) sequencing kit includes:
hybridizing oligos to the endonuclease restriction sites, after optical imaging;
using endonucleases to fragment the nanoballs,
wherein the nanoballs contain capture sequences or fragments; and
allowing the fragments to be captured by the beads in the Chromium sc-RNA-seq kit.

13. The method as claimed in claim 5, wherein the sequencing of the nanoballs is performed using a 10×'s Chromium fixed ribonucleic acid (RNA) protocol and the protocol includes:
hybridizing a pair of deoxyribonucleic acid (DNA) oligos to the nanoballs and subsequently ligating together;
wherein the ligated oligos contain a segment with a unique sequence corresponding to the nanoball that each probe hybridizes to; and
allowing the ligated oligos to be sequenced following the 10×'s chromium fixed ribonucleic acid (RNA) protocol.

14. The method as claimed in claim 5, wherein the nanoballs include one or a plurality of unique molecular identifiers (UMI).

15. The method as claimed in claim 14, wherein the unique molecular identifier (UMI) serves as a molecular tag that enables accurate counting of the nanoballs during sequencing.

16. The method as claimed in claim 15, wherein the method further comprises incorporating one or a plurality of unique molecular identifiers during counting; a plurality of identical nanoballs is distinguished and counted as separate entities, allowing for correlation between the microscopy count and the sequencing count of the non-unique nanoballs.

17. The method as claimed in claim 5, wherein the imaging of the nanoballs is performed by employing a color combinatorial coding approach.

18. The method as claimed in claim 5, wherein the nanoballs are generated in situ using multiple rounds of hybridization.

19. The method of claim 18, wherein the multiple rounds of hybridization for generating the nanoballs further include:
staining a first set of oligos with nucleic acid or deoxyribonucleic acid (DNA) oligo-tagged antibodies in a sample;
hybridizing a second set of oligos to the first plurality, with the second set being specific to the first set;
subsequently hybridizing a third set of oligos to the second plurality and reiterating the process for a predetermined number of iterations.

20. The method as claimed in claim 5, wherein the nanoballs are readout using single cell combinatorial indexing RNA sequencing (sci-RNA-seq) kit when the nanoballs include restriction sites, and the process comprises:
hybridization of the nanoballs containing a polyA sequence to a reverse-transcription primer (RT-primer);
using a hairpin oligo to ligate to the reverse-transcription primer (RT-primer);
performing elongation using the reverse-transcription primer (RT-primer) as a template; and
incorporating the single cell combinatorial indexing RNA sequencing (sci-RNA-seq) barcodes and enabling the generation of the single cell combinatorial indexing RNA sequencing (sci-RNA-seq) library for the nanoball readout.
